(19) United States
(12) Patent Application Publication
(10) Pub. No.: US 2013/0178653 A1
(43) Pub. Date: Jul. 11, 2013

(54) NOVEL PRECURSOR OF RADIOLABELLED CHOLINE ANALOG COMPOUNDS

(71) Applicants: Eric Ofori Aboagye, London (GB); Edward George Robins, Singapore (SG); Graham Smith, London (GB); Yongjun Zhao, Jiang Su Province (CN)

(72) Inventors: Eric Ofori Aboagye, London (GB); Edward George Robins, Singapore (SG); Graham Smith, London (GB); Yongjun Zhao, Jiang Su Province (CN)

(73) Assignees: IMPERIAL COLLEGE, SOUTH KENSINGTON (GB); GE HEALTHCARE LIMITED, LITTLE CHALFONT (GB)

(21) Appl. No.: 13/824,438

(22) PCT Filed: Sep. 20, 2011
(86) PCT No.: PCT/US11/52239

§ 371 Date: Mar. 18, 2013

Related U.S. Application Data
(60) Provisional application No. 61/384,891, filed on Sep. 21, 2010.

(51) Int. Cl. C07C 215/08; C07C 217/10

(52) U.S. Cl. CPC C07C 215/08; C07C 217/10
USPC 564/346; 564/503

(57) ABSTRACT

The present invention describes intermediate(s), pre-cursor(s), for the preparation of radialabelled choline analogs to be used as radiotracers for Positron Emission Tomography (PET) or Single Photon Emission Computed Tomography (SPECT) imaging of diseases.

### FIELD OF THE INVENTION

The present invention describes a novel radiotracer(s) for Positron Emission Tomography (PET) or Single Photon Emission Computed Tomography (SPECT) imaging of disease states related to altered choline metabolism (e.g., tumor imaging of prostate, breast, brain, esophageal, ovarian, endometrial, lung and prostate cancer primary tumor, nodal disease or metastases). The present invention also describes intermediate(s), precursor(s), pharmaceutical composition(s), methods of making, and methods of use of the novel radiotracer(s).

### DESCRIPTION OF RELATED ART

The biosynthetic product of choline kinase (EC 2.7.1.32) activity, phosphocholine, is elevated in several cancers and is a precursor for membrane phosphatidylcholine (Aboagye, E. O., et al., Cancer Res 1999; 59:80-4; Exton, J. H., Biochim Biophys Acta 1994; 1212:26-42; George, T. P., et al., Biochim Biophys Acta 1989; 104:283-91; and Teegarden, D., et al., J Biol Chem 1990; 265(11):6042-7). Over-expression of choline kinase and increased enzyme activity have been reported in prostate, breast, lung, ovarian and colon cancers (Aoyama, C., et al., Prog Lipid Res 2004; 43(3):266-81; Glunde, K., et al., Cancer Res 2004; 64(12):4270-6; Glunde, K., et al., Cancer Res 2005; 65(23): 11034-43; Iorio, E., et al., Cancer Res 2005; 65(20): 9369-76; Ramirez de Molina, A., et al., Biochem Biophys Res Commun 2002; 296(3): 580-3; and Ramirez de Molina, A., et al., Lancet Oncol 2007; 8(10): 889-97) and are largely responsible for the increased phosphocholine levels with malignant transformation and progression; the increased phosphocholine levels in cancer cells are also due to increased breakdown via phospholipase C (Glunde, K., et al., Cancer Res 2004; 64(12):4270-6).

Because of this phenotype, together with reduced urinary excretion, [¹¹C]choline has become a prominent radiotracer for positron emission tomography (PET) and PET-Computed Tomography (PET-CT) imaging of prostate cancer, and to a lesser extent imaging of brain, esophageal, and lung cancer (Hara, T., et al., J Nucl Med 2000; 41:1507-13; Hara, T., et al., J Nucl Med 1998; 39:990-5; Hara, T., et al., J Nucl Med 1997; 38:842-7; Kobori, 0., et al., Cancer Cell 1999; 86:1638-48; Pieterman, R. M., et al., J Nucl Med 2002; 43(2):167-72; and Reske, S. N. Eur J Nucl Med Mol Imaging 2008; 35:1741). The specific PET signal is due to transport and phosphorylation of the radiotracer to [¹¹C]phosphocholine by choline kinase.

Of interest, however, is that [¹¹C]choline (as well as the fluoro-analog) is oxidized to [¹¹C]betaine by choline oxidase (see FIG. 1 below) (EC 1.1.3.17) mainly in kidney and liver tissues, with metabolites detectable in plasma soon after injection of the radiotracer (Roivainen, A., et al., European Journal of Nuclear Medicine 2000; 27:25-32). This makes discrimination of the relative contributions of parent radiotracer and catabolites difficult when a late imaging protocol is used.

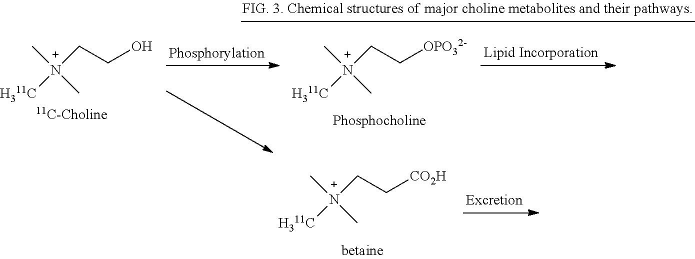

[¹⁸F]Fluoromethylcholine ([¹⁸F]FCH):

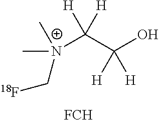

was developed to overcome the short physical half-life of carbon-11 (20.4 min) (DeGrado, T. R., et al., Cancer Res 2001; 61(1):110-7) and a number of PET and PET-CT studies with this relatively new radiotracer have been published (Beheshti, M., et al., Eur J Nucl Med Mol Imaging 2008; 35(10):1766-74; Cimitan, M., et al., Eur J Nucl Med Mol Imaging 2006; 33(12):1387-98; de Jong, I. J., et al., Eur J Nucl Med Mol Imaging 2002; 29:1283-8; and Price, D. T., et al., J Urol 2002; 168(1):273-80). The longer half-life of fluorine-18 (109.8 min) was deemed potentially advantageous in permitting late imaging of tumors when sufficient clearance of parent tracer in systemic circulation had occurred (DeGrado, T. R., et al., J Nucl Med 2002; 43(1):92-6).

WO2001/82864 describes 18F-labeled choline analogs, including [18F]Fluoromethylcholine ([18F]-FCH) and their use as imaging agents (e.g., PET) for the non-invasive detection and localization of neoplasms and pathophysiologies influencing choline processing in the body (Abstract). WO2001/82864 also describes 18F-labeled di-deuterated choline analogs such as [¹⁸F]fluoromethyl-[1-²H₂]choline ([¹⁸F]FDC)(hereinafter referred to as “[¹⁸F]D2-FCH”):

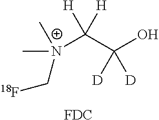

The oxidation of choline under various conditions; including the relative oxidative stability of choline and [1,2-²H₄]choline has been studied (Fan, F., et al., Biochemistry 2007, 46, 6402-6408; Fan, F., et al., Journal of the American Chemical Society 2005, 127, 2067-2074; Fan, F., et al., Journal of the American Chemical Society 2005, 127, 17954-17961; Gadda, G. Biochimica et Biophysica Acta 2003, 1646, 112-118; Gadda, G., Biochimica et Biophysica Acta 2003, 1650, 4-9). Theoretically the effect of the extra deuterium substitution was found to be neglible in the context of a primary isotope effect of 8-10 since the β-secondary isotope effect is ˜1.05 (Fan, F., et al., Journal of the American Chemical Society 2005, 127, 17954-17961).

[¹⁸F]Fluoromethylcholine is now used extensively in the clinic to image tumour status (Beheshti, M., et al., Radiology 2008, 249, 389-90; Beheshti, M., et al., Eur J Nucl Med Mol Imaging 2008, 35, 1766-74).

The present invention, as described below, provides a novel precursor compound. These novel precursor compounds can be used in the synthesis of, for example, ¹⁸F-radiolabeled radiotracers which in turn that can be used for PET imaging of choline metabolism.

### BRIEF DESCRIPTION OF THE DRAWINGS

FIG. 1 depicts the chemical structures of major choline metabolites and their pathways.

FIG. 3 shows NMR analysis of tetradeuterated choline precursor. Top, ¹H NMR spectrum; bottom, ¹³C NMR spectrum. Both spectra were acquired in CDCl₃.

FIG. 4 depicts the HPLC profiles for the synthesis of [¹⁸F]fluoromethyl tosylate (9) and [¹⁸F]fluoromethyl-[1,2-²H₄]choline (D4-FCH) showing (A) radio-HPLC profile for synthesis of (9) after 15 mins; (B) UV (254 nm) profile for synthesis of (9) after 15 mins; (C) radio-HPLC profile for synthesis of (9) after 10 mins; (D) radio-HPLC profile for crude (9); (E) radio-HPLC profile of formulated (9) for injection; (F) refractive index profile post formulation (cation detection mode).

FIG. 5a is a picture of a fully assembled cassette of the present invention for the production of [¹⁸F]fluoromethyl-[1,2-²H₄]choline (D4-FCH) via an unprotected precursor.

FIG. 5b is a picture of a fully assembled cassette of the present invention for the production of [¹⁸F]fluoromethyl-[1,2-²H₄]choline (D4-FCH) via a PMB-protected precursor.

FIG. 6 depicts representative radio-HPLC analysis of potassium permanganate oxidation study. Top row are control samples for [¹⁸F]fluoromethylcholine ([¹⁸F]FCH) and [¹⁸F]fluoromethyl-[1,2-²H₄]choline ([¹⁸F]D4-FCH), extracts from the reaction mixture at time zero (0 min) Bottom row are extracts after treatment for 20 mins Left hand side are for [¹⁸F]fluoromethylcholine ([¹⁸F]FCH), right are for [¹⁸F] fluoromethyl-[1,2-²H₄]choline ([¹⁸F]D4-FCH).

FIG. 7 shows chemical oxidation potential of [¹⁸F]fluoromethylcholine and [¹⁸F]fluoromethyl-[1,2-²H₄]choline in the presence of potassium permanganate.

FIG. 8 shows time-course stability assay of [¹⁸F]fluoromethylcholine and [¹⁸F]fluoromethyl-[1,2-²H₄]choline in the presence of choline oxidase demonstrating conversion of parent compounds to their respective betaine analogues.

FIG. 9 shows representative radio-HPLC analysis of choline oxidase study. Top row are control samples for [¹⁸F]fluoromethylcholine and [¹⁸F]fluoromethyl-[1,2-²H₄]choline, extracts from the reaction mixture at time zero (0 min) Bottom row are extracts after treatment for 40 mins Left hand side are of [¹⁸F]fluoromethylcholine, right are of [¹⁸F]fluoromethyl-[1,2-²H₄]choline.

FIG. 10. Top: Analysis of the metabolism of [¹⁸F]fluoromethylcholine (FCH) to [¹⁸F]FCH-betaine and [¹⁸F]fluoromethyl-[1,2-²H₄]choline (D4-FCH) to [¹⁸F]D4-FCH-betaine by radio-HPLC in mouse plasma samples obtained 15 min after injecting the tracers i.v. into mice. Bottom: summary of the conversion of parent tracers, [¹⁸F]fluoromethylcholine (FCH) and [¹⁸F]fluoromethyl-[1,2-²H₄]choline (D4-FCH), to metabolites, [¹⁸F]FCH-betaine (FCHB) and [¹⁸F]D4-FCH betaine (D4-FCHB), in plasma.

FIG. 11. Biodistribution time course of [¹⁸F]fluoromethylcholine (FCH), [¹⁸F]fluoromethyl-[1-²H₂]choline (D2-FCH) and [¹⁸F]fluoromethyl-[1,2-²H₄]choline (D4-FCH) in HCT-116 tumor bearing mice. Inset: the time points selected for evaluation. A) Biodistribution of [¹⁸F]fluoromethylcholine; B) biodistribution of [¹⁸F]fluoromethyl-[1-²H₂]choline; C) biodistribution of [¹⁸F]fluoromethyl-[1,2-²H₄]choline; D) time course of tumor uptake for [¹⁸F]fluoromethylcholine (FCH), [¹⁸F]fluoromethyl-[1-²H₂]choline (D2-FCH) and [¹⁸F]fluoromethyl-[1,2-²H₄]choline (D4-FCH) from charts A-C. Approximately 3.7 MBq of [¹⁸F]fluoromethylcholine (FCH), [¹⁸F]fluoromethyl-[1-²H₂]choline (D2-FCH) and [¹⁸F]fluoromethyl-[1,2-²H₄]choline (D4-FCH) injected into awake male C3H-Hej mice which were sacrificed under isofluorane anesthesia at the indicated time points.

FIG. 12 shows radio-HPLC chromatograms to show distribution of choline radiotracer metabolites in tissue harvested from normal white mice at 30 min p.i. Top row, radiotracer standards; middle row, kidney extracts; bottom row, liver extracts. On the left is [¹⁸F]FCH, on the right [¹⁸F]D4-FCH.

FIG. 13 show radio-HPLC chromatograms to show metabolite distribution of choline radiotracers in HCT116 tumors 30 min post-injection. Top-row, neat radiotracer standards; bottom row, 30 min tumor extracts. Left side, [¹⁸F]FCH; middle, [¹⁸F]D4-FCH; right, [¹¹C]choline.

FIG. 14 shows radio-HPLC chromatograms for phosphocholine HPLC validation using HCT116 cells. Left, neat [¹⁸F]FCH standard; middle, phosphatase enzyme incubation; right, control incubation.

FIG. 15 shows distribution of radiometabolites for [¹⁸F]fluoromethylcholine analogs: ¹⁸F]fluoromethylcholine, [¹⁸F]fluoromethyl-[1-²H₂]choline and [¹⁸F]fluoromethyl-[1,2-²H₄]choline at selected time points.

FIG. 16 shows tissue profile of [¹⁸F]FCH and [¹⁸F]D4-FCH. (a) Time versus radioactivity curve for the uptake of [¹⁸F]FCH in liver, kidney, urine (bladder) and muscle derived from PET data, and (b) corresponding data for [¹⁸F]D4-FCH. Results are the mean±SE; n=4 mice. For clarity upper and lower error bars (SE) have been used. (Leyton, et al., Cancer Res 2009: 69:(19), pp 7721-7727).

FIG. 17 shows tumor profile of [¹⁸F]FCH and [¹⁸F]D4-FCH in SKMEL28 tumor xenograft. (a) Typical [¹⁸F]FCH-PET and [¹⁸F]D4-FCH-PET images of SKMEL28 tumor-bearing mice showing 0.5 mm transverse sections through the tumor and coronal sections through the bladder. For visualization, 30 to 60 min summed image data are displayed. Arrows point to the tumors (T), liver (L) and bladder (B). (b). Comparison of time versus radioactivity curves for [¹⁸F]FCH and [¹⁸F]D4-FCH in tumors. For each tumor, radioactivity at each of 19 time frames was determined. Data are mean % ID/vox₆₀ mean±SE (n=4 mice per group). (c) Summary of imaging variables. Data are mean±SE, n=4; *P=0.04. For clarity upper and lower error bars (SE) have been used.

FIG. 18 shows the effect of PD0325901, a mitogenic extracellular kinase inhibitor, on uptake of [¹⁸F]D4-FCH in HCT116 tumors and cells. (a) Normalized time versus radioactivity curves in HCT116 tumors following daily treatment for 10 days with vehicle or 25 mg/kg PD0325901. Data are the mean±SE; n=3 mice. (b) Summary of imaging variables % ID/vox₆₀, % ID/vox₆₀ₘₐₓ, and AUC. Data are mean±SE; * P=0.05.(c) Intrinsic cellular effect of PD0325901 (1 μM) on [¹⁸F]D4-FCH phosphocholine metabolism after treating HCT116 cells for 1 hr with [¹⁸F]D4-FCH in culture. Data are mean±SE; n=3; * P=0.03.

FIG. 19 shows expression of choline kinase A in HCT116 tumors. (a) A typical Western blot demonstrating the effect of PD0325901 on tumor choline kinase A (CHKA) protein expression. HCT116 tumors from mice that were injected with PD0325901 (25 mg/kg daily for 10 days, orally) or vehicle were analyzed for CHKA expression by western blotting. β-actin was used as the loading control. (b) Summary densitometer measurements for CHKA expression expressed as a ratio to β-actin. The results are the mean ratios±SE; n=3, * P=0.05.

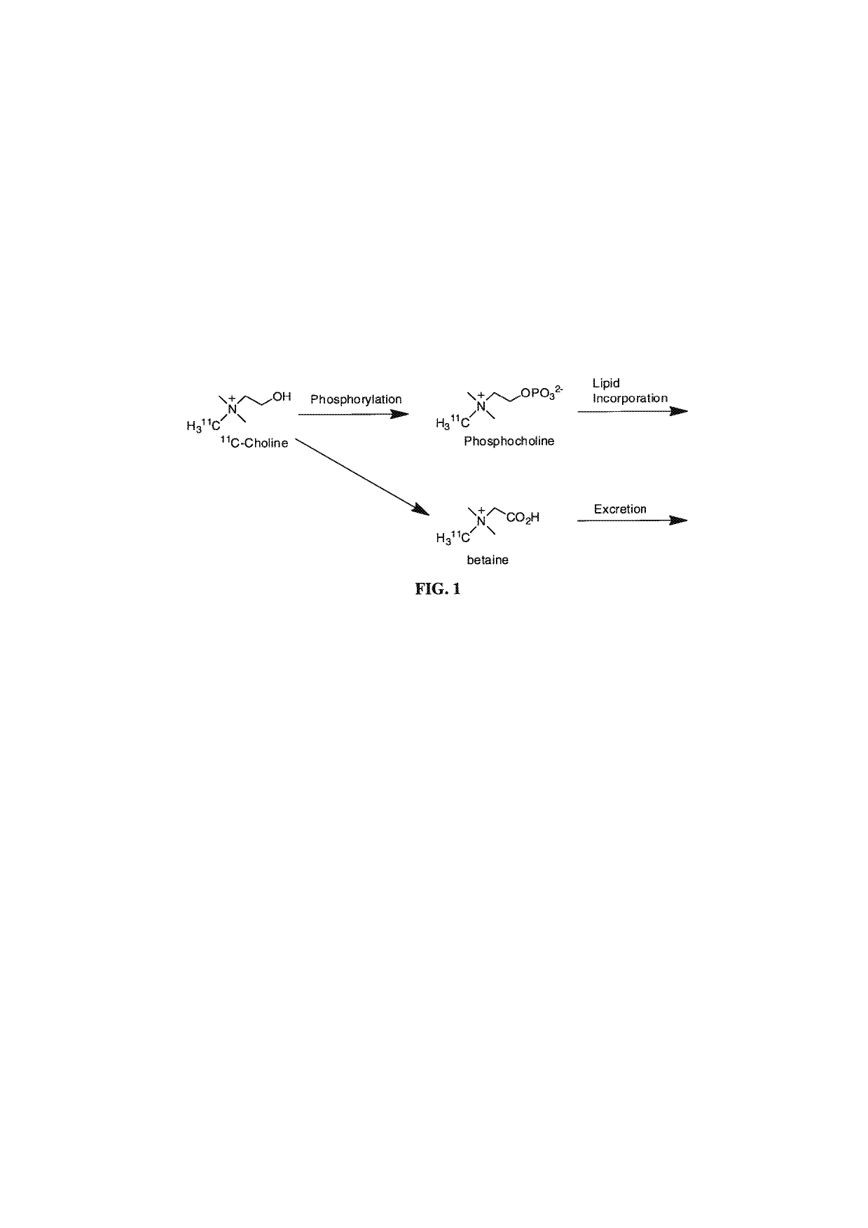

### SUMMARY OF THE INVENTION

The present invention further provides a precursor compound of Formula (II):

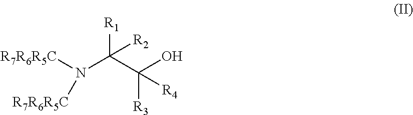

wherein:

R₁, R₂, R₃, and R₄ are each independently hydrogen or deuterium (D);

R₅, R₆, and R₇ are each independently hydrogen, R₈, —(CH₂)ₘR₈, —(CD₂)ₘR₈, —(CF₂)ₘR₈, —CH(R₈)₂, or —CD(R₈)₂;

R₈ is independently hydrogen, —OH, —CH₃, —CF₃, —CH₂OH, —CH₂F, —CH₂Cl, —CH₂Br, —CH₂I, —CD₃, —CD₂OH, —CD₂F, CD₂Cl, CD₂Br, CD₂I, or —C₆H₅; and

m is an integer from 1-4.

The present invention further provides a method of making a precursor compound of Formula (II).

The present invention further provides a pharmaceutical composition comprising a precursor compound of Formula (II) and a pharmaceutically acceptable carrier or excipient.

### DETAILED DESCRIPTION OF THE INVENTION

The present invention provides a novel radiolabeled choline analog compound of formula (I):

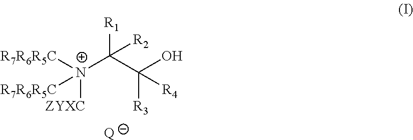

wherein:

R₁, R₂, R₃, and R₄ are each independently hydrogen or deuterium (D);

R₅, R₆, and R₇ are each independently hydrogen, R₈, —(CH₂)ₘR₈, —(CD₂)ₘR₈, —(CF₂)ₘR₈, —CH(R₈)₂, or —CD(R₈)₂;

R₈ is independently hydrogen, —OH, —CH₃, —CF₃, —CH₂OH, —CH₂F, —CH₂Cl, —CH₂Br, —CH₂I, —CD₃, —CD₂OH, —CD₂F, CD₂Cl, CD₂Br, CD₂I, or —C₆H₅;

m is an integer from 1-4;

X and Y are each independently hydrogen, deuterium (D), or F;

Z is a halogen selected from F, Cl, Br, and I or a radioisotope; and

Q is an anionic counterion;

with the proviso that said compound of formula (I) is not fluoromethylcholine, fluoromethyl-ethyl-choline, fluoromethyl-propyl-choline, fluoromethyl-butyl-choline, fluoromethyl-pentyl-choline, fluoromethyl-isopropyl-choline, fluoromethyl-isobutyl-choline, fluoromethyl-sec-butyl-choline, fluoromethyl-diethyl-choline, fluoromethyl-diethanol-choline, fluoromethyl-benzyl-choline, fluoromethyl-triethanol-choline, 1,1-dideuterofluoromethylcholine, 1,1-dideuterofluoromethyl-ethyl-choline, 1,1-dideuterofluoromethyl-propyl-choline, or an [¹⁸F] analog thereof.

In a preferred embodiment of the invention, a compound of Formula (I) is provided wherein:

R₁, R₂, R₃, and R₄ are each independently hydrogen;

R₅, R₆, and R₇ are each independently hydrogen, R₈, —(CH₂)ₘR₈, —(CD₂)ₘR₈, —(CF₂)ₘR₈, —CH(R₈)₂, or —CD(R₈)₂;

R₈ is independently hydrogen, —OH, —CH₃, —CF₃, —CH₂OH, —CH₂F, —CH₂Cl, —CH₂Br, —CH₂I, —CD₃, —CD₂OH, —CD₂F, CD₂Cl, CD₂Br, CD₂I, or —C₆H₅;

m is an integer from 1-4;

X and Y are each independently hydrogen, deuterium (D), or F;

Z is a halogen selected from F, Cl, Br, and I or a radioisotope;

Q is an anionic counterion;

with the proviso that said compound of formula (I) is not fluoromethylcholine, fluoromethyl-ethyl-choline, fluoromethyl-propyl-choline, fluoromethyl-butyl-choline, fluoromethyl-pentyl-choline, fluoromethyl-isopropyl-choline, fluoromethyl-isobutyl-choline, fluoromethyl-sec-butyl-choline, fluoromethyl-diethyl-choline, fluoromethyl-diethanol-choline, fluoromethyl-benzyl-choline, fluoromethyl-triethanol-choline, or an [¹⁸F] analog thereof.

In a preferred embodiment of the invention, a compound of Formula (I) is provided wherein:

R₁ and R₂ are each hydrogen;

R₃ and R₄ are each deuterium (D);

R₅, R₆, and R₇ are each independently hydrogen, R₈, —(CH₂)ₘR₈, —(CD₂)ₘR₈, —(CF₂)ₘR₈, —CH(R₈)₂, or —CD(R₈)₂;

R₈ is independently hydrogen, —OH, —CH₃, —CF₃, —CH₂OH, —CH₂F, —CH₂Cl, —CH₂Br, —CH₂I, —CD₃, —CD₂OH, —CD₂F, CD₂Cl, CD₂Br, CD₂I, or —C₆H₅;

m is an integer from 1-4;

X and Y are each independently hydrogen, deuterium (D), or F;

Z is a halogen selected from F, Cl, Br, and I or a radioisotope;

Q is an anionic counterion;

with the proviso that said compound of formula (I) is not 1,1-dideuterofluoromethylcholine, 1,1-dideuterofluoromethyl-ethyl-choline, 1,1-dideuterofluoromethyl-propyl-choline, or an [¹⁸F] analog thereof.

In a preferred embodiment of the invention, a compound of Formula (I) is provided wherein:

R₁, R₂, R₃, and R₄ are each deuterium (D);

R₅, R₆, and R₇ are each independently hydrogen, R₈, —(CH₂)ₘR₈, —(CD₂)ₘR₈, —(CF₂)ₘR₈, —CH(R₈)₂, or —CD(R₈)₂;

R₈ is independently hydrogen, —OH, —CH₃, —CF₃, —CH₂OH, —CH₂F, —CH₂Cl, —CH₂Br, —CH₂I, —CD₃, —CD₂OH, —CD₂F, CD₂Cl, CD₂Br, CD₂I, or —C₆H₅;

m is an integer from 1-4;

X and Y are each independently hydrogen, deuterium (D), or F;

Z is a halogen selected from F, Cl, Br, and I or a radioisotope;

Q is an anionic counterion.

According to the present invention, when Z of a compound of Formula (I) as described herein is a halogen, it can be a halogen selected from F, Cl, Br, and I; preferably, F.

According to the present invention, when Z of a compound of Formula (I) as described herein is a radioisotope (hereinafter referred to as a “radiolabeled compound of Formula (I)”), it can be any radioisotope known in the art. Preferably, Z is a radioisotope suitable for imaging (e.g., PET, SPECT). More preferably Z is a radioisotope suitable for PET imaging. Even more preferably, Z is ¹⁸F, ⁷⁶Br, ¹²³I, ¹²⁴I, or ¹²⁵I. Even more preferably, Z is ¹⁸F.

According to the present invention, Q of a compound of Formula (I) as described herein can be any anionic counterion known in the art suitable for cationic ammonium compounds. Suitable examples of Q include anionic: bromide (Br⁻), chloride (CP), acetate (CH₃CH₂C(O)O⁻), or tosylate (⁻OTos). In a preferred embodiment of the invention, Q is bromide (Br⁻) or tosylate (−OTos). In a preferred embodiment of the invention, Q is chloride (Cl⁻) or acetate (CH₃CH₂C(O)O⁻). In a preferred embodiment of the invention, Q is chloride (Cl⁻).

According the invention, a preferred embodiment of a compound of Formula (I) is the following compound of Formula (Ia):

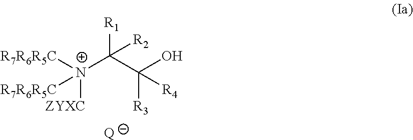

wherein:

R₁, R₂, R₃, and R₄ are each independently deuterium (D);

R₅, R₆, and R₇ are each hydrogen;

X and Y are each independently hydrogen;

Z is ¹⁸F;

Q is CF.

According to the invention, a preferred compound of Formula (Ia) is [¹⁸F]fluoromethyl-[1,2-²H₄]-choline ([¹⁸F]-D4-FCH). [¹⁸F]-D4-FCH is a more metabolically stable fluorocholine (FCH) analog. [¹⁸F]-D4-FCH offers numerous advantages over the corresponding 18F-non-deuterated and/or 18F-di-deuterated analog. For example, [¹⁸F]-D4-FCH exhibits increased chemical and enzymatic oxidative stability relative to [¹⁸F]fluoromethylcholine. [¹⁸F]-D4-FCH has an improved in vivo profile (i.e., exhibits better availability for in vivo imaging) relative to dideuterofluorocholine, [¹⁸F]fluoromethyl-[1-²H₂]choline, that is over and above what could be predicted by literature precedence and is, thus, unexpected. [¹⁸F]D4-FCH exhibits improved stability and consequently will better enable late imaging of tumors after sufficient clearance of the radiotracer from systemic circulation. [¹⁸F]-D4-FCH also enhances the sensitivity of tumor imaging through increased availability of substrate. These advantages are discussed in further detail below.

The present invention provides a compound of formula (III):

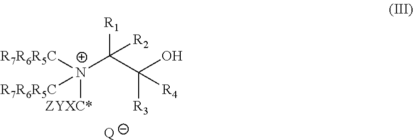

wherein:

R₁, R₂, R₃, and R₄ are each independently hydrogen or deuterium (D);

R₅, R₆, and R₇ are each independently hydrogen, R₈, —(CH₂)ₘR₈, —(CD₂)ₘR₈, —(CF₂)ₘR₈, —CH(R₈)₂, or —CD(R₈)₂;

R₈ is independently hydrogen, —OH, —CH₃, —CF₃, —CH₂OH, —CH₂Cl, —CH₂Br, —CH₂I, —CD₃, —CD₂OH, CD₂Cl, CD₂Br, CD₂I, or —C₆H₅;

m is an integer from 1-4;

C* is a radioisotope of carbon;

X, Y and Z are each independently hydrogen, deuterium (D), a halogen selected from F, Cl, Br, and I, alkyl, alkenyl, alkynl, aryl, heteroaryl, heterocyclyl group; and

Q is an anionic counterion; with the proviso the compound of Formula (III) is not ¹¹C-choline.

According to the invention, C* of the compound of formula (III) can be any radioisotope of carbon. Suitable examples of C* include, but are not limited to, ¹¹C, ¹³C, and ¹⁴C. Q is a described for the compound of Formula (I).

In a preferred embodiment of the invention, a compound of Formula (III) is provided wherein C* is ¹¹C; X and Y are each hydrogen; and Z is F.

### Pharmaceutical or Radiopharmaceutical Composition

The present invention provides a pharmaceutical or radiopharmaceutical composition comprising a compound for Formula (I), including a compound of Formula (Ia), each as defined herein together with a pharmaceutically acceptable carrier, excipient, or biocompatible carrier. According to the invention when Z of a compound of Formula (I) or (Ia) is a radioisotope, the pharmaceutical composition is a radiopharmaceutical composition.

The present invention further provides a pharmaceutical or radiopharmaceutical composition comprising a compound for Formula (I), including a compound of Formula (Ia), each as defined herein together with a pharmaceutically acceptable carrier, excipient, or biocompatible carrier suitable for mammalian administration.

The present invention provides a pharmaceutical or radiopharmaceutical composition comprising a compound for Formula (III), as defined herein together with a pharmaceutically acceptable carrier, excipient, or biocompatible carrier.

The present invention further provides a pharmaceutical or radiopharmaceutical composition comprising a compound for Formula (III), as defined herein together with a pharmaceutically acceptable carrier, excipient, or biocompatible carrier suitable for mammalian administration.

As would be understood by one of skill in the art, the pharmaceutically acceptable carrier or excipient can be any pharmaceutically acceptable carrier or excipient known in the art.

The “biocompatible carrier” can be any fluid, especially a liquid, in which a compound of Formula (I), (Ia), or (III) can be suspended or dissolved, such that the pharmaceutical composition is physiologically tolerable, e.g., can be administered to the mammalian body without toxicity or undue discomfort. The biocompatible carrier is suitably an injectable carrier liquid such as sterile, pyrogen-free water for injection; an aqueous solution such as saline (which may advantageously be balanced so that the final product for injection is either isotonic or not hypotonic); an aqueous solution of one or more tonicity-adjusting substances (e.g., salts of plasma cations with biocompatible counterions), sugars (e.g., glucose or sucrose), sugar alcohols (e.g., sorbitol or mannitol), glycols (e.g., glycerol), or other non-ionic polyol materials (e.g., polyethyleneglycols, propylene glycols and the like). The biocompatible carrier may also comprise biocompatible organic solvents such as ethanol. Such organic solvents are useful to solubilise more lipophilic compounds or formulations. Preferably the biocompatible carrier is pyrogen-free water for injection, isotonic saline or an aqueous ethanol solution. The pH of the biocompatible carrier for intravenous injection is suitably in the range 4.0 to 10.5.

The pharmaceutical or radiopharmaceutical composition may be administered parenterally, i.e., by injection, and is most preferably an aqueous solution. Such a composition may optionally contain further ingredients such as buffers; pharmaceutically acceptable solubilisers (e.g., cyclodextrins or surfactants such as Pluronic, Tween or phospholipids); pharmaceutically acceptable stabilisers or antioxidants (such as ascorbic acid, gentisic acid or para-aminobenzoic acid). Where a compound of Formula (I), (Ia), or (III) is provided as a radiopharmaceutical composition, the method for preparation of said compound may further comprise the steps required to obtain a radiopharmaceutical composition, e.g., removal of organic solvent, addition of a biocompatible buffer and any optional further ingredients. For parenteral administration, steps to ensure that the radiopharmaceutical composition is sterile and apyrogenic also need to be taken. Such steps are well-known to those of skill in the art.

### Preparation of a Compound of the Invention

The present invention provides a method to prepare a compound for Formula (I), including a compound of Formula (Ia), wherein said method comprises reaction of the precursor compound of Formula (II) with a compound of Formula (Ma) to form a compound of Formula (I) (Scheme A):

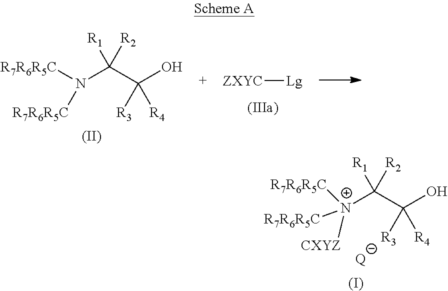

wherein the compounds of Formulae (I) and (II) are each as described herein and the compound of Formula (Ma) is as follows:

ZXYC-Lg (IIIa)

wherein X, Y and Z are each as defined herein for a compound of Formula (I) and “Lg” is a leaving group. Suitable examples of “Lg” include, but are not limited to, bromine (Br) and tosylate (OTos). A compound of Formula (Ma) can be prepared by any means known in the art including those described herein.

Synthesis of a compound of Formula (Ma) wherein Z is F; X and Y are both H and the Lg is OTos (i.e., fluoromethyltosylate) can be achieved as set forth in Scheme 3 below:

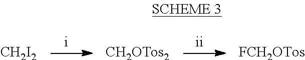

wherein: i: Silver p-toluenesulfonate, MeCN, reflux, 20 h;
ii: KF, MeCN, reflux, 1 h.
   According to Scheme 3 above:

### (a) Synthesis of Methylene Ditosylate

Commercially available diiodomethane can be reacted with silver tosylate, using the method of Emmons and Ferris, to give methylene ditosylate (Emmons, W. D., et al., “Metathetical Reactions of Silver Salts in Solution. II. The Synthesis of Alkyl Sulfonates”, Journal of the American Chemical Society, 1953; 75:225).

(b) Synthesis of cold Fluoromethyltosylate

Fluoromethyltosylate can be prepared by nucleophilic substitution of Methylene ditosylate from step (a) using potassium fluoride/Kryptofix K₂₂₂ in acetonitrile at 80° C. under standard conditions.

When Z is a radioisotope, the radioisotope can be introduced by any means known by one of skill in the art. For example, the radioisotope [¹⁸F]-fluoride ion (¹⁸F⁻) is normally obtained as an aqueous solution from the nuclear reaction ¹⁸O(p,n)¹⁸F and is made reactive by the addition of a cationic counterion and the subsequent removal of water. Suitable cationic counterions should possess sufficient solubility within the anhydrous reaction solvent to maintain the solubility of 18F⁻. Therefore, counterions that have been used include large but soft metal ions such as rubidium or caesium, potassium complexed with a cryptand such as Kryptofix™, or tetraalkylammonium salts. A preferred counterion is potassium complexed with a cryptand such as Kryptofix™ because of its good solubility in anhydrous solvents and enhanced ¹⁸F⁻ reactivity. ¹⁸F can also be introduced by nucleophilic displacement of a suitable leaving group such as a halogen or tosylate group. A more detailed discussion of well-known ¹⁸F labelling techniques can be found in Chapter 6 of the “Handbook of Radiopharmaceuticals” (2003; John Wiley and Sons: M. J. Welch and C. S. Redvanly, Eds.). For example, [18F]Fluoromethyltosylate can be prepared by nucleophilic substitution of Methylene ditosylate with [¹⁸F]-fluoride ion in acetonitrile containing 2-10% water (see Neal, T. R., et al., Journal of Labelled Compounds and Radiopharmaceuticals 2005; 48:557-68).

### Automated Synthesis

In a preferred embodiment, the method to prepare a compound for Formula (I), including a compound of Formula (Ia), is automated. For example, [¹⁸F]-radiotracers may be conveniently prepared in an automated fashion by means of an automated radiosynthesis apparatus. There are several commercially-available examples of such platform apparatus, including TRACERlab™ (e.g., TRACERlab™ MX) and FASTlab™ (both from GE Healthcare Ltd.). Such apparatus commonly comprises a “cassette”, often disposable, in which the radiochemistry is performed, which is fitted to the apparatus in order to perform a radiosynthesis. The cassette normally includes fluid pathways, a reaction vessel, and ports for receiving reagent vials as well as any solid-phase extraction cartridges used in post-radiosynthetic clean up steps. Optionally, in a further embodiment of the invention, the automated radiosynthesis apparatus can be linked to a high performance liquid chromatograph (HPLC).

The present invention therefore provides a cassette for the automated synthesis of a compound of Formula (I), including a compound of Formula (Ia), each as defined herein comprising:
i) a vessel containing the precursor compound of Formula (II) as defined herein; and
ii) means for eluting the contents of the vessel of step (i) with a compound of Formula (Ma) as defined herein.
   For the cassette of the invention, the suitable and preferred embodiments of the precursor compound of Formulae (II) and (Ma) are each as defined herein.

In one embodiment of the invention, a method of making a compound of Formula (I), including a compound of Formula (Ia), each as described herein, that is compatible with FASTlab™ from a protected ethanolamine precursor that requires no HPLC purification step is provided.

The radiosynthesis of [¹⁸F]fluoromethyl-[1,2-²H₄]choline (¹⁸F-D4-FCH) can be performed according to the methods and examples described herein. The radiosynthesis of ¹⁸F-D4-FCH can also be performed using commercially available synthesis platforms including, but not limited to, GE FASTlab™ (commercially available from GE Healthcare Inc.).

An example of a FASTlab™ radiosynthetic process for the preparation of [¹⁸F]fluoromethyl-[1,2-²H₄]choline from a protected precursor is shown in Scheme 5:

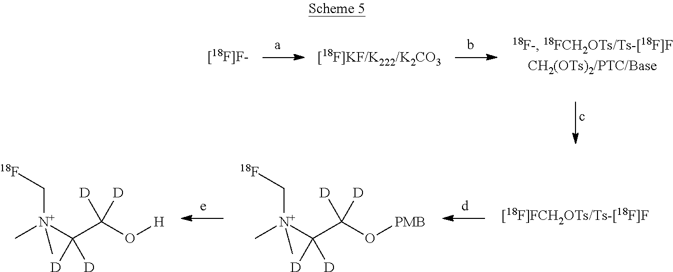

wherein:
a. Preparation of [¹⁸F]KF/K₂₂₂/K₂CO₃ complex as described in more detail below;
b. Preparation of [¹⁸F]FCH₂OTs as described in more detail below;
c. SPE purification of [¹⁸F]FCH₂OTs as described in more detail below;
d. Radiosynthesis of O-PMB-[¹⁸F]-D₄-Choline (O-PMB-[¹⁸F]D4-FCH) as described in more detail below; and
e. Purification & formulation of [¹⁸F]D₄-Choline (¹⁸F-D4-FCH) as the hydrochloric salt as described in more detail below.

The automation of [¹⁸F]fluoro-[1,2-²H₄]choline or [¹⁸F]fluorocholine (from the protected precursor) involves an identical automated process (and are prepared from the fluoromethylation of O-PMB-N,N-dimethyl-[1,2-²H₄]ethanolamine and O-PMB-N,N-dimethylethanolamine respectively).

According to one embodiment of the present invention, FASTlab™ syntheses of [¹⁸F]fluoromethyl-[1,2-²H₄]choline or [¹⁸F]fluoromethylcholine comprises the following sequential steps:

(i) Trapping of [¹⁸F]fluoride onto QMA;
(ii) Elution of [¹⁸F]fluoride from a QMA;
(iii) Radiosynthesis of [¹⁸F]FCH₂OTs;
(iv) SPE clean up of [¹⁸F]FCH₂OTs;
(v) Reaction vessel clean up;
(vi) Drying reaction vessel and [¹⁸F]fluoromethyl tosylate retained on SPE t-C18 plus simultaneously;
(vii) Alkylation reaction;
(viii) Removal of unreacted O-PMB-precursor; and
(ix) Deprotection & formulation.

Each of steps (i)-(ix) are described in more detail below.

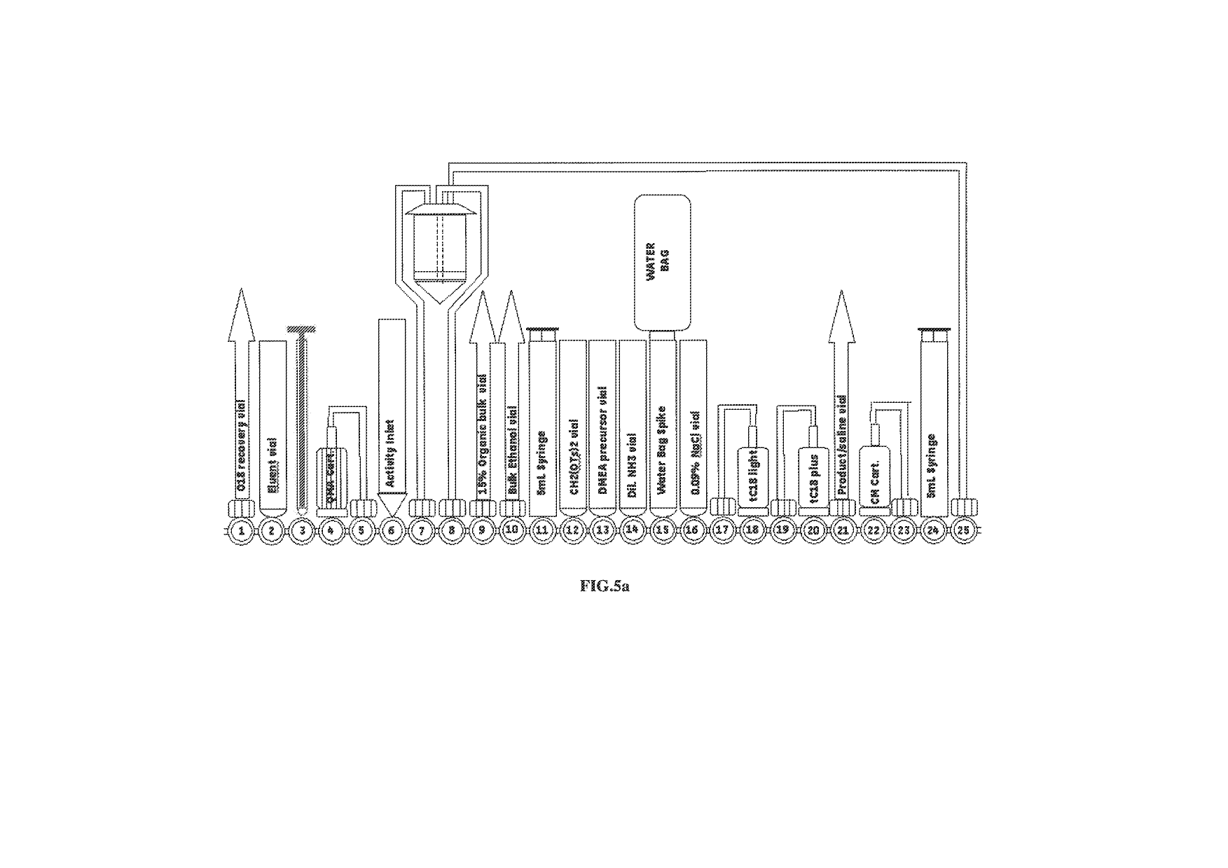

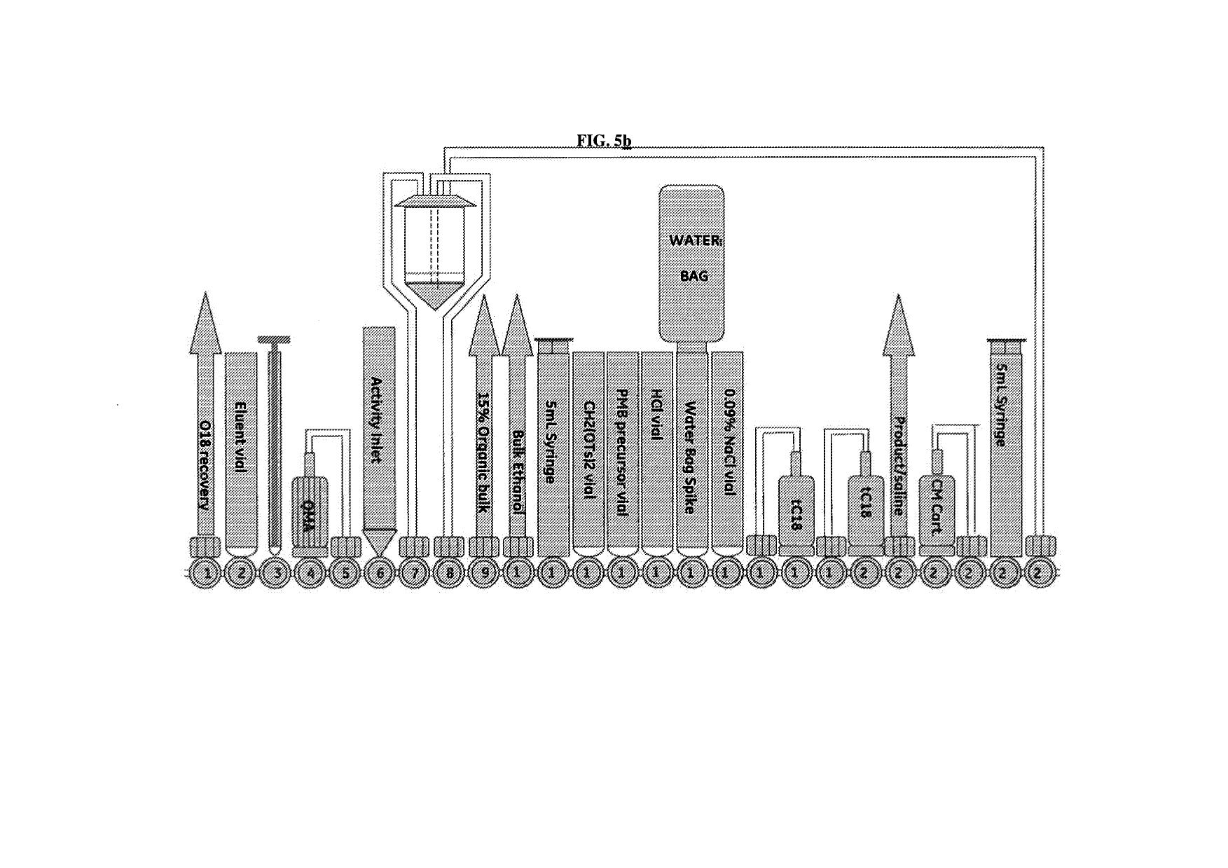

In one embodiment of the present invention, steps (i)-(ix) above are performed on a cassette as described herein. One embodiment of the present invention is a cassette capable of performing steps (i)-(ix) for use in an automated synthesis platform. One embodiment of the present invention is a cassette for the radiosynthesis of [¹⁸F]fluoromethyl-[1,2-²H₄]choline ([¹⁸F]D4-FCH) or [¹⁸F]fluoromethylcholine from a protected precursor. An example of a cassette of the present invention is shown in FIG. 5b.

(i) Trapping of [¹⁸F]Fluoride onto QMA

[¹⁸F]fluoride (typically in 0.5 to 5 mL H₂¹⁸O) is passed through a pre-conditioned Waters QMA cartridge.

(ii) Elution of [¹⁸F]Fluoride from a QMA

The eluent, as described in Table 1 is withdrawn into a syringe from the eluent vial and passed over the Waters QMA into the reaction vessel. This procedure elutes [¹⁸F]fluoride into the reaction vessel. Water and acetonitrile are removed using a well-designed drying cycle of “nitrogen/vacuum/heating/cooling”.

(iii) Radiosynthesis of [¹⁸F]FCH₂OTs

Once the K[¹⁸F]Fluoride/K222/K₂CO₃ complex of (ii) is dry, CH₂(OTs)₂ methylene ditosylate in a solution containing acetonitrile and water is added to the reaction vessel containing the K[¹⁸F]fluoride/K222/K₂CO₃ complex. The resulting reaction mixture will be heated (typically to 110° C. for 10 min), then cooled down (typically to 70° C.).

### (iv) SPE Clean up of [¹⁸F]FCH₂OTs

Once radiosynthesis of [¹⁸F]FCH₂OTs is completed and the reaction vessel is cooled, water is added into the reaction vessel to reduce the organic solvent content in the reaction vessel to approximately 25%. This diluted solution is transferred from the reaction vessel and through the t-C18-light and t-C18 plus cartridges—these cartridges are then rinsed with 12 to 15 mL of a 25% acetonitrile/75% water solution. At the end of this process:
the methylene ditosylate remains trapped on the t-C18-light and
the [¹⁸F]FCH₂OTs, tosyl-[¹⁸F]fluoride remains trapped on the t-C18 plus.

### (v) Reaction Vessel Clean Up

The reaction vessel was cleaned (using ethanol) prior to the alkylation of [¹⁸F]fluoroethyl tosylate and O-PMB-DMEA precursor.

(vi) Drying Reaction Vessel and [¹⁸F]Fluoromethyl Tosylate Retained on SPE t-C18 Plus Simultaneously

Once clean up (v) was completed, the reaction vessel and the [¹⁸F]fluoromethyl tosylate retained on SPE t-C18 plus was dried simultaneously.

(vii) Alkylation Reaction

Following step (vi), the [¹⁸F]FCH₂OTs (along with tosyl-[¹⁸F]fluoride) retained on the t-C18 plus was eluted into the reaction vessel using a mixture of O-PMB-N,N-dimethyl-[1,2-²H₄]ethanolamine (or O-PMB-N,N-dimethylethanolamine) in acetonitrile.

The alkylation of [¹⁸F]FCH₂OTs with O-PMB-precursor was achieved by heating the reaction vessel (typically 110° C. for 15 min) to afford [¹⁸F]fluoro-[1,2-²H₄]choline (or O-PMB-[¹⁸F]fluorocholine).

(viii) Removal of Unreacted O-PMB-Precursor

Water (3 to 4 mL) was added to the reaction and this solution was then passed through a pre-treated CM cartridge, followed by an ethanol wash—typically 2×5 mL (this removes unreacted O-PMB-DMEA) leaving “purified” [¹⁸F]fluoro-[1,2-²H₄]choline (or O-PMB-[¹⁸F]fluorocholine) trapped onto the CM cartridge.

### (ix) Deprotection & Formulation

Hydrochloric acid was passed through the CM cartridge into a syringe: this resulted in the deprotection of O-PMB-[¹⁸F]fluorocholine (the syringe contains [¹⁸F]fluorocholine in a HCl solution). Sodium acetate was then added to this syringe to buffer to pH 5 to 8 affording D4-choline (or [¹⁸F]choline) in an acetate buffer. This buffered solution is then transferred to a product vial containing a suitable buffer.

Table 1 provides a listing of reagents and other components required for preparation of [¹⁸F]fluoromethyl-[1,2-²H₄]choline (D4-FCH) (or [¹⁸F]fluoromethylcholine) radiocassette of the present invention:

TABLE 1

| Reagent/Component | Description |
|---|---|
| Eluents | Eluent contains either: |
| | K₂₂₂/K₂CO₃ water/acetonitrile or |
| | K₂₂₂/KHCO₃ water/acetonitrile or |
| | 18-crown-6/K₂CO₃ water/acetonitrile or |
| | 18-crown-6/KHCO₃ water/acetonitrile. |
| 25% acetonitrile/75% water | 5 mL acetonitrile/15 mL water. |
| Ethanol | 35 mL of ethanol |
| CH₂(OTs)₂ | methylene ditosylate in an aqueous |
| | acetonitrile solution |
| t-C18 light | SPE cartridge commercially available from |
| | Waters (Milford, MA, USA) |
| | Preconditioned by passing acetonitrile and |
| | water (2 mL each) through |
| CM light | Commercially available from Waters |
| cartridge | (Milford, MA, USA). Preconditioned by |
| | passing through 1M hydrochloric acid and |
| | water (2 mL each). |
| PMB-O-precursor | O-PMB-N,N-dimethyl-[1,2- |
| | ²H₄]ethanolamine and O-PMB-N,N- |
| | dimethylethanolamine in anhydrous |
| | acetonitrile |
| HCl | hydrochloric acid [1 to 5M] |
| NaOAC | sodium acetate solution [1 to 5M] |
| Water bag | 100 mL water |
| t-C18 plus | SPE cartridge commercially available from |
| | Waters (Milford, MA, USA) |
| | Preconditioned by passing acetonitrile and |
| | water (2 mL each) through |
| Ion exchange cartridge | Water pre-conditioned QMA light carb |
| | commercially available from Waters |
| | (Milford, MA, USA) |

According to one embodiment of the present invention, FASTlab™ synthesis of [¹⁸F]fluoromethyl-[1,2-²H₄]choline via an unprotected precursor comprises the following sequential steps as depicted in Scheme 6 below:

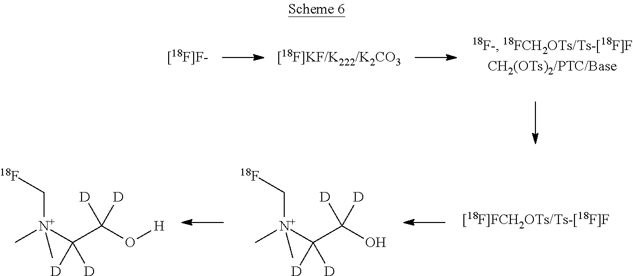

1. Recovery of [¹⁸F]fluoride from QMA;

2. Preparation of K[¹⁸F]F/K₂₂₂/K₂CO₃ complex;

3. Radiosynthesis of ¹⁸FCH₂OTs;

4. SPE cleanup of ¹⁸FCH₂OTs;

5. Clean up of reaction vessel cassette and syringe;

6. Drying of reaction vessel and C18 SepPak;

7. Elution off and coupling of ¹⁸FCH₂OTs with D4-DMEA;

8. Transfer of reaction mixture onto CM cartridge;

9. Clean up of cassette and syringe;

10. Washing of CM cartridge with dilute aq ammonia solution, Ethanol and water;

11. Elution of [¹⁸F]fluoromethyl-[1,2-²H₄]choline from CM cartridge with 0.09% sodium chloride (5 ml), followed by water (5 ml).

In one embodiment of the present invention, steps (1)-(11) above are performed on a cassette as described herein. One embodiment of the present invention is a cassette capable of performing steps (1)-(11) for use in an automated synthesis platform. One embodiment of the present invention is a cassette for the radiosynthesis of [¹⁸F]fluoromethyl-[1,2-²H₄]choline ([¹⁸F]D4-FCH) from an unprotected precursor. An example of a cassette of the present invention is shown in FIG. 5a.

Table 2 provides a listing of reagents and other components required for preparation of [¹⁸F]fluoromethyl-[1,2-²H₄]choline (D4-FCH) (or [¹⁸F]fluoromethylcholine) via an unprotected precursor radiocassette of the present invention:

TABLE 2

| Reagent/Component | Description |
|---|---|
| Sep-Pak light QMA | Commercially available from Waters |
| Carbonate cartridge | (Milford, MA, USA). Used as supplied. |
| Eluent prepared from stock | K₂CO₃: 17.9 mg/ml in water: 200 ul. |
| solutions: | Kryptofix222: 12 mg/ml in acetonitrile: |
| | 800 ul. |
| Organic wash for C18 | 15% acetonitrile in water, preloaded into |
| Sep-Pak pair | vial. |
| Bulk ethanol | 50 ml preloaded into vial |
| CH₂(OTs)₂ | 4.4 mg of methylene ditosylate dissolved |
| | into 1.25 ml acetonitrile containing 2% |
| | water. Solution pre-loaded into vial. |
| t-C18 Sep-Pak light | SPE cartridge commercially available |
| | from Waters (Milford, MA, USA). |
| | Preconditioned by passing acetonitrile |
| | then water through. |
| t-C18 Sep-Pak Plus | SPE cartridge commercially available |
| | from Waters (Milford, MA, USA). |
| | Preconditioned by passing acetonitrile |
| | then water through. |
| Deuterated | Custom synthesis. 150-200 ul dissolved |
| dimethylethanolamine | into 1.4 ml acetonitrile. Preloaded into |
| | vial. |
| Water bag | 100 ml bag of sterile purified water. |
| Aqueous ammonia solution | 10-15 ul of concentrated (30%) |
| | ammonia in 10 ml water. 4 ml of this |
| | solution preloaded into vial. |
| Sep-Pak light CM cartridge | Cartridge commercially available from |
| | Waters (Milford, MA, USA). Used as |
| | supplied. |
| Sodium Chloride for | 0.09% sodium chloride solution prepared |
| product formulation | from 0.9% sodium chloride BP and water |
| | for injection. BP. |

### Imaging Method

The radiolabeled compound of the invention, as described herein, will be taken up into cells via cellular transporters or by diffusion. In cells where choline kinase is overexpressed or activated the radiolabeled compound of the invention, as described herein, will be phosphorylated and trapped within that cell. This will form the primary mechanism of detecting neoplastic tissue.

The present invention further provides a method of imaging comprising the step of administering a radiolabeled compound of the invention or a pharmaceutical composition comprising a radiolabeled compound of the invention, each as described herein, to a subject and detecting said radiolabeled compound of the invention in said subject. The present invention further provides a method of detecting neoplastic tissue in vivo using a radiolabeled compound of the invention or a pharmaceutical composition comprising a radiolabeled compound of the invention, each as described herein. Hence the present invention provides better tools for early detection and diagnosis, as well as improved prognostic strategies and methods to easily identify patients that will respond or not to available therapeutic treatments. As a result of the ability of a compound of the invention to detect neoplastic tissue, the present invention further provides a method of monitoring therapeutic response to treatment of a disease state associated with the neoplastic tissue.

In a preferred embodiment of the invention, the radiolabeled compound of the invention for use in a method of imaging of the invention, as described herein, is a radiolabeled compound of Formula (I).

In a preferred embodiment of the invention, the radiolabeled compound of the invention for use in a method of imaging of the invention, as described herein, is a radiolabeled compound of Formula (III).

As would be understood by one of skill in the art the type of imaging (e.g., PET, SPECT) will be determined by the nature of the radioisotope. For example, if the radiolabeled compound of Formula (I) contains ¹⁸F it will be suitable for PET imaging.

Thus the invention provides a method of detecting neoplastic tissue in vivo comprising the steps of:
i) administering to a subject a radiolabeled compound of the invention or a pharmaceutical composition comprising a radiolabeled compound of the invention, each as defined herein;
ii) allowing said a radiolabeled compound of the invention to bind neoplastic tissue in said subject;
iii) detecting signals emitted by said radioisotope in said bound radiolabeled compound of the invention;
iv) generating an image representative of the location and/or amount of said signals; and,
v) determining the distribution and extent of said neoplastic tissue in said subject.

The step of “administering” a radiolabeled compound of the invention is preferably carried out parenterally, and most preferably intravenously. The intravenous route represents the most efficient way to deliver the compound throughout the body of the subject. Intravenous administration neither represents a substantial physical intervention nor a substantial health risk to the subject. The radiolabeled compound of the invention is preferably administered as the radiopharmaceutical composition of the invention, as defined herein. The administration step is not required for a complete definition of the imaging method of the invention. As such, the imaging method of the invention can also be understood as comprising the above-defined steps (ii)-(v) carried out on a subject to whom a radiolabeled compound of the invention has been pre-administered.

Following the administering step and preceding the detecting step, the radiolabeled compound of the invention is allowed to bind to the neoplastic tissue. For example, when the subject is an intact mammal, the radiolabeled compound of the invention will dynamically move through the mammal's body, coming into contact with various tissues therein. Once the radiolabeled compound of the invention comes into contact with the neoplastic tissue it will bind to the neoplastic tissue.

The “detecting” step of the method of the invention involves detection of signals emitted by the radioisotope comprised in the radiolabeled compound of the invention by means of a detector sensitive to said signals, e.g., a PET camera. This detection step can also be understood as the acquisition of signal data.

The “generating” step of the method of the invention is carried out by a computer which applies a reconstruction algorithm to the acquired signal data to yield a dataset. This dataset is then manipulated to generate images showing the location and/or amount of signals emitted by the radioisotope. The signals emitted directly correlate with the amount of enzyme or neoplastic tissue such that the “determining” step can be made by evaluating the generated image.

The “subject” of the invention can be any human or animal subject. Preferably the subject of the invention is a mammal. Most preferably, said subject is an intact mammalian body in vivo. In an especially preferred embodiment, the subject of the invention is a human.

The “disease state associated with the neoplastic tissue” can be any disease state that results from the presence of neoplastic tissue. Examples of such disease states include, but are not limited to, tumors, cancer (e.g., prostate, breast, lung, ovarian, pancreatic, brain and colon). In a preferred embodiment of the invention the disease state associated with the neoplastic tissue is brain, breast, lung, espophageal, prostate, or pancreatic cancer.

As would be understood by one of skill in the art, the “treatment” will be depend on the disease state associated with the neoplastic tissue. For example, when the disease state associated with the neoplastic tissue is cancer, treatment can include, but is not limited to, surgery, chemotherapy and radiotherapy. Thus a method of the invention can be used to monitor the effectiveness of the treatment against the disease state associated with the neoplastic tissue.

Other than neoplasms, a radiolabeled compound of the invention may also be useful in liver disease, brain disorders, kidney disease and various diseases associated with proliferation of normal cells. A radiolabeled compound of the invention may also be useful for imaging inflammation; imaging of inflammatory processes including rheumatoid arthritis and knee synovitis, and imaging of cardiovascular disease including artherosclerotic plaque.

### Precursor Compound

The present invention provides a precursor compound of Formula (II):

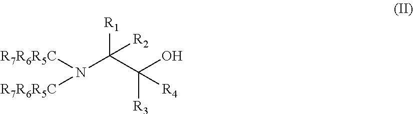

as described above.

In a preferred embodiment of the invention, a compound of Formula (II) is provided wherein:

R₁, R₂, R₃, and R₄ are each independently hydrogen;

R₅, R₆, and R₇ are each independently hydrogen, R₈, —(CH₂)ₘR₈, —(CD₂)ₘR₈, —(CF₂)ₘR₈, or —CD(R₈)₂;

R₈ is hydrogen, —OH, —CH₃, —CF₃, —CH₂OH, —CH₂F, —CH₂Cl, —CH₂Br, —CH₂I, —CD₃, —CD₂OH, —CD₂F, CD₂Cl, CD₂Br, CD₂I, or —C₆H₅; and

m is an integer from 1-4.

In a preferred embodiment of the invention, a compound of Formula (II) is provided wherein:

R₁ and R₂ are each hydrogen;

R₃ and R₄ are each deuterium (D);

R₅, R₆, and R₇ are each independently hydrogen, R₈, —(CH₂)ₘR₈, —(CD₂)ₘR₈, —(CF₂)ₘR₈, or —CD(R⁸)₂;

R₈ is hydrogen, —OH, —CH₃, —CF₃, —CH₂OH, —CH₂F, —CH₂Cl, —CH₂Br, —CH₂I, —CD₃, —CD₂OH, —CD₂F, CD₂Cl, CD₂Br, CD₂I, or —C₆H₅; and

m is an integer from 1-4.

In a preferred embodiment of the invention, a compound of Formula (II) is provided wherein:

R₁, R₂, R₃, and R₄ are each deuterium (D);

R₅, R₆, and R₇ are each independently hydrogen, R₈, —(CH₂)ₘR₈, —(CD₂)ₘR₈, —(CF₂)ₘR₈, or —CD(R₈)₂;

R₈ is hydrogen, —OH, —CH₃, —CF₃, —CH₂OH, —CH₂F, —CH₂Cl, —CH₂Br, —CH₂I, —CD₃, —CD₂OH, —CD₂F, CD₂Cl, CD₂Br, CD₂I, or —C₆H₅; and

m is an integer from 1-4.

According to the invention, compound of Formula (II) is a compound of Formula (IIa):

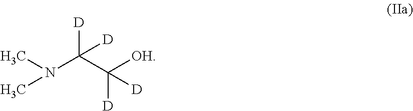

In one embodiment of the invention, a compound of Formula (IIb) is provided:

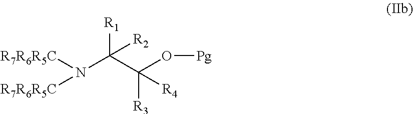

wherein:

R₁, R₂, R₃, and R₄ are each independently hydrogen or deuterium (D);

R₅, R₆, and R₇ are each independently hydrogen, R⁸, —(CH₂)ₘR₈, —(CD₂)ₘR₈, —(CF₂)ₘR₈, —CH(R⁸)₂, or —CD(R⁸)₂;

R₈ is independently hydrogen, —OH, —CH₃, —CF₃, —CH₂OH, —CH₂F, —CH₂Cl, —CH₂Br, —CH₂I, —CD₃, —CD₂OH, —CD₂F, CD₂Cl, CD₂Br, CD₂I, or —C₆H₅; and

m is an integer from 1-4; and

Pg is a hydroxyl protecting group.

In a preferred embodiment of the invention, a compound of Formula (IIb) is provided wherein Pg is a p-methoxybenyzl (PMB), trimethylsilyl (TMS), or a dimethoxytrityl (DMTr) group.

In a preferred embodiment of the invention, a compound of Formula (Hb) is provided wherein Pg is a p-methoxybenyzl (PMB) group.

In one embodiment of the invention, a compound of Formula (Bc) is provided:

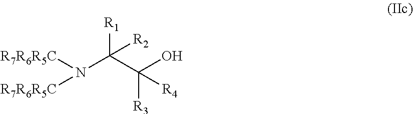

wherein:

R₁, R₂, R₃, and R₄ are each independently hydrogen or deuterium (D);

R₅, R₆, and R₇ are each independently hydrogen, R⁸, —(CH₂)ₘR₈, —(CD₂)ₘR₈, —(CF₂)ₘR₈, —CH(R⁸)₂, or —CD(R⁸)₂;

R₈ is independently hydrogen, —OH, —CH₃, —CF₃, —CH₂OH, —CH₂F, —CH₂Cl, —CH₂Br, —CH₂I, —CD₃, —CD₂OH, —CD₂F, CD₂Cl, CD₂Br, CD₂I, or —C₆H₅; and

m is an integer from 1-4;

with the proviso that when R₁, R₂, R₃, and R₄ are each hydrogen, R₅, R₆, and R₇ are each not hydrogen; and with the proviso that when R₁, R₂, R₃, and R₄ are each deuterium, R₅, R₆, and R₇ are each not hydrogen.

In a preferred embodiment of the invention, a compound of Formula (IIc) is provided wherein:

R₁, R₂, R₃, and R₄ are each independently hydrogen;

R₅, R₆, and R₇ are each independently hydrogen, R₈, —(CH₂)ₘR₈, —(CD₂)ₘR₈, —(CF₂)ₘR₈, or —CD(R₈)₂;

R₈ is hydrogen, —OH, —CH₃, —CF₃, —CH₂OH, —CH₂F, —CH₂Cl, —CH₂Br, —CH₂I, —CD₃, —CD₂OH, —CD₂F, CD₂Cl, CD₂Br, CD₂I, or —C₆H₅; and

m is an integer from 1-4; with the proviso that R₅, R₆, and R₇ are each not hydrogen.

In a preferred embodiment of the invention, a compound of Formula (IIc) is provided wherein:

R₁, R₂, R₃, and R₄ are each deuterium (D);

R₅, R₆, and R₇ are each independently hydrogen, R₈, —(CH₂)ₘR₈, —(CD₂)ₘR₈, —(CF₂)ₘR₈, or —CD(R₈)₂;

R₈ is hydrogen, —OH, —CH₃, —CF₃, —CH₂OH, —CH₂F, —CH₂Cl, —CH₂Br, —CH₂I, —CD₃, —CD₂OH, —CD₂F, CD₂Cl, CD₂Br, CD₂I, or —C₆H₅; and

m is an integer from 1-4; with the proviso that R₅, R₆, and R₇ are each not hydrogen.

In a preferred embodiment of the invention, a compound of Formula (IIc) is provided wherein:

R₁ and R₂ are each hydrogen; and

R₃ and R₄ are each deuterium (D).

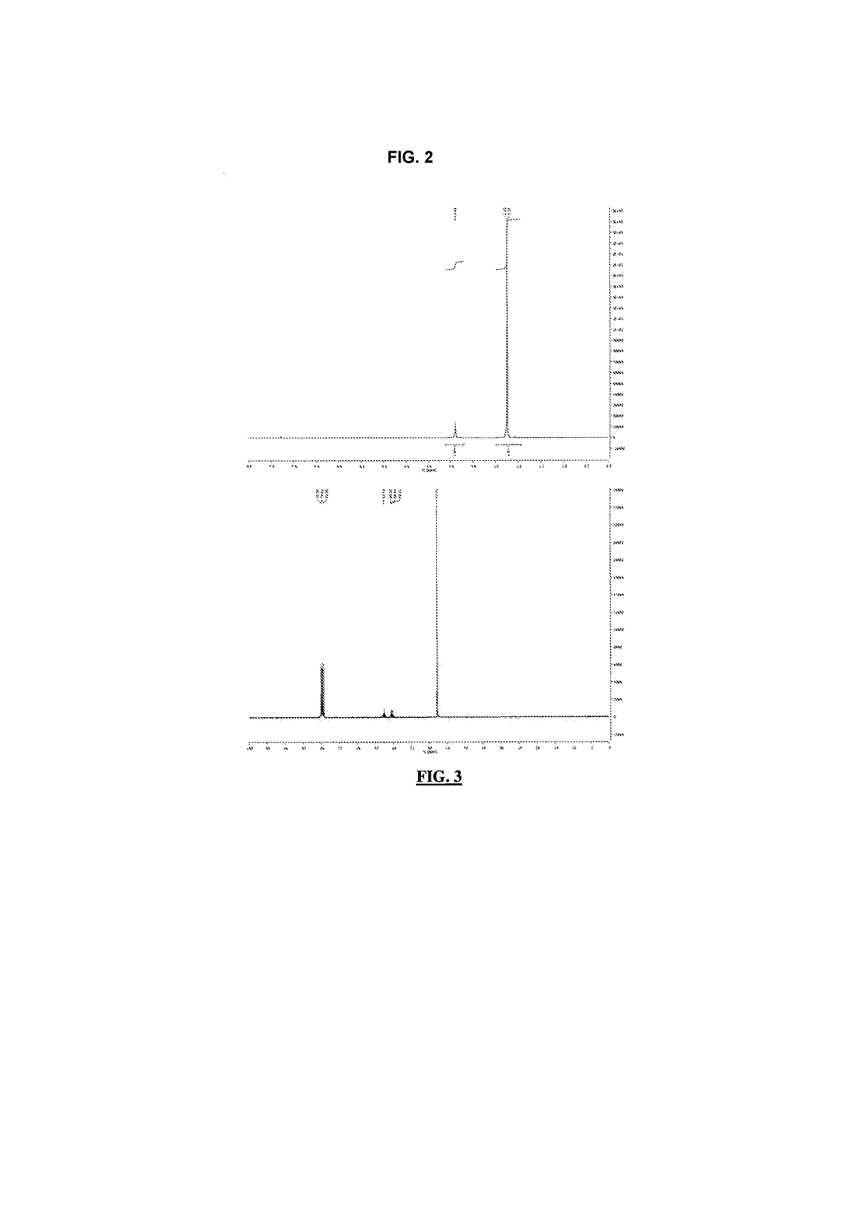

A precursor compound of Formula (II), including a compound of Formula (IIa), (IIb) and (IIc), can be prepared by any means known in the art including those described herein. For example, the compound of Formula (IIa) can be synthesized by alkylation of dimethylamine in THF with 2-bromoethanol-1,1,2,2-d₄ in the presence of potassium carbonate as shown in Scheme 1 below:

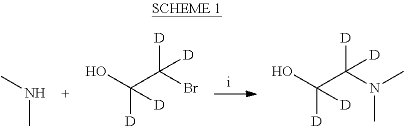

wherein i=K₂CO₃, THF, 50° C., 19 h. The desired tetra-deuterated product can be purified by distillation. The ¹H NMR spectrum of the compound of Formula (IIa) (FIG. 3) in deuteriochloroform showed only the peaks associated with the N,N-dimethyl groups and the hydroxyl of the alcohol; no peaks associated with the hydrogens of the methylene groups of the ethyl alcohol chain were observed. Consistent with this, the ¹³C NMR spectrum (FIG. 3) showed the large singlet associated with the N,N-dimethyl carbons; however, the peaks for the ethyl alcohol methylene carbons at 60.4 ppm and 62.5 ppm were substantially reduced in magnitude, suggesting the absence of the signal enhancement associated with the presence of a covalent carbon-hydrogen bond. In addition, the methylene peaks are both split into multiplets, indicating spin-spin coupling. Since ¹³C NMR is typically run with ¹H decoupling, the observed multiplicity must be the result of carbon-deuterium bonding. On the basis of the above observations the isotopic purity of the desired product is considered to be >98% in favour of the ²H isotope (relative to the ¹H isotope).

A di-deuterated analog of a precursor compound of Formula (II) can be synthesized from N,N-dimethylglycine via lithium aluminium hydride reduction as shown in Scheme 2 below:

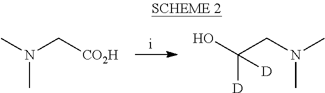

wherein i=LiAlD₄, THF, 65° C., 24 h. ¹³C NMR analysis indicated that isotopic purity of greater than 95% in favor of the ²H isomer (relative to the ¹H isotope) can be achieved.

According to the invention, the hydroxyl group of a compound of Formula (II), including a compound of Formula (IIa) can be further protected with a protecting group to give a compound of Formula (IIb):

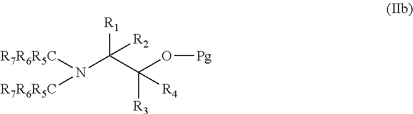

wherein Pg is any hydroxyl protecting group known in the art. Preferably, Pg is any acid labile hydroxyl protecting group including, for example, those described in “”Protective Groups in Organic Synthesis”, 3rd Edition, A Wiley Interscience Publication, John Wiley & Sons Inc., Theodora W. Greene and Peter G. M. Wuts, pp 17-200. Preferably, Pg is a p-methoxybenzyl (PMB), trimethylsilyl (TMS), or a dimethoxytrityl (DMTr) group. More preferably, Pg is a p-methoxybenyzl (PMB) group.
Validation of [¹⁸F]fluoromethyl-1,2-²H₄ choline (D4-FCH)

Stability to oxidation resulting from isotopic substitution was evaluated in in vitro chemical and enzymatic models using [¹⁸F]fluoromethylcholine as standard. [¹⁸F]Fluoromethyl-[1,2-²H₄]choline was then evaluated in in vivo models and compared to [¹¹C]choline, [¹⁸F]fluoromethylcholine and [¹⁸F]Fluoromethyl-[1-²H₂]choline:

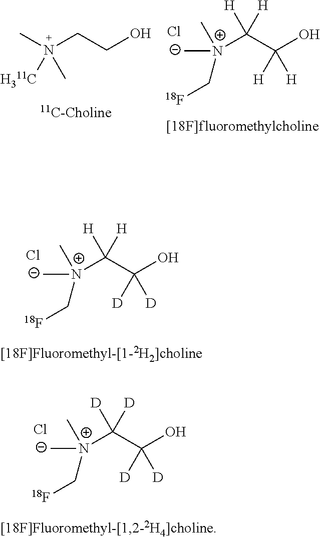

Potassium Permanganate oxidation study

The effect of deuterium substitution on bond strength was initially tested by evaluation of the chemical oxidation pattern of [¹⁸F]fluoromethylcholine and [¹⁸F]Fluoromethyl-[1,2-²H₄]choline using potassium permanganate. Scheme 6 below details the base catalyzed potassium permanganate oxidation of [¹⁸F]fluoromethylcholine and [¹⁸F]Fluoromethyl-[1,2-²H₄]choline at room temperature, with aliquots removed and analyzed by radio-HPLC at pre-selected time points:

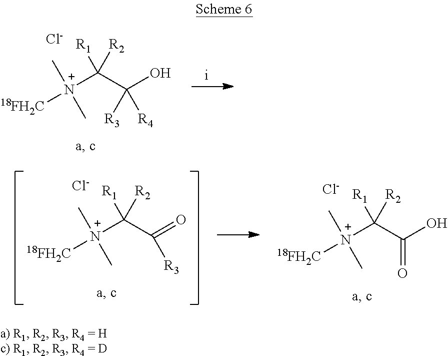

### Reagents and Conditions: i) KMnO₄, Na₂CO₃, H₂O, rt.

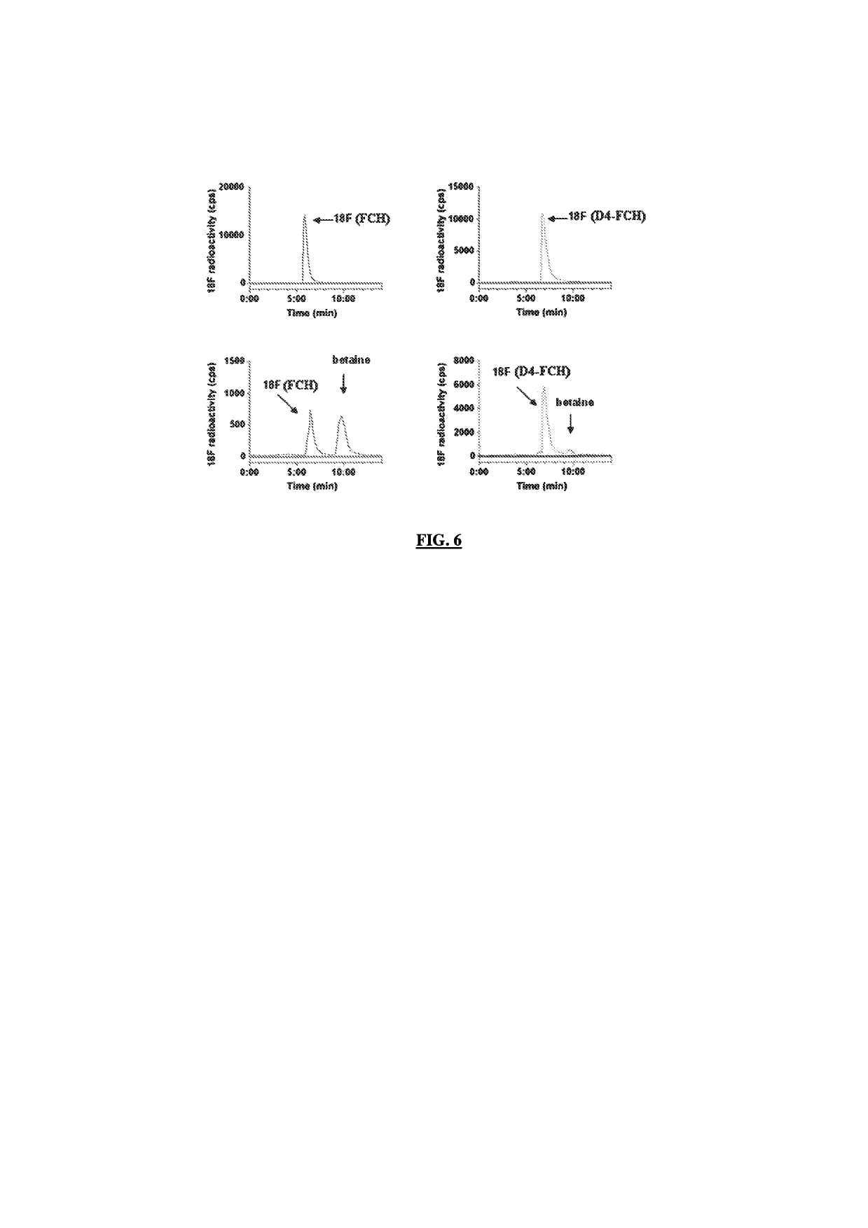

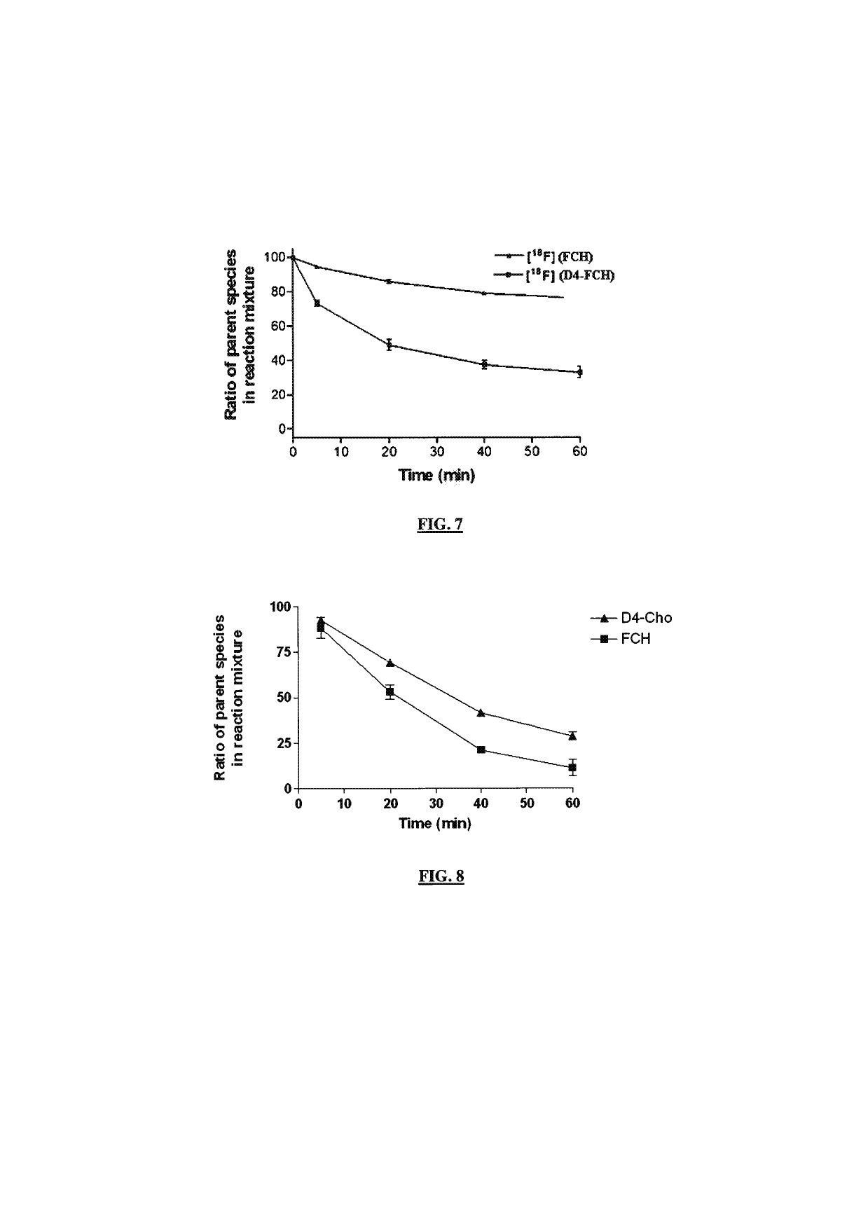

The results are summarized in FIGS. 6 and 7. The radio-HPLC chromatogram (FIG. 6) showed a greater proportion of the parent compound remaining at 20 min for [¹⁸F]Fluoromethyl-[1,2-²H₄]choline. The graph in FIG. 7 further showed a significant isotope effect for the deuterated analogue, [¹⁸F]Fluoromethyl-[1,2-²H₄]choline, with nearly 80% of parent compound still present 1 hour post-treatment with potassium permanganate, compared to less than 40% of parent compound [¹⁸F]Fluoromethylcholine still present at the same time point.

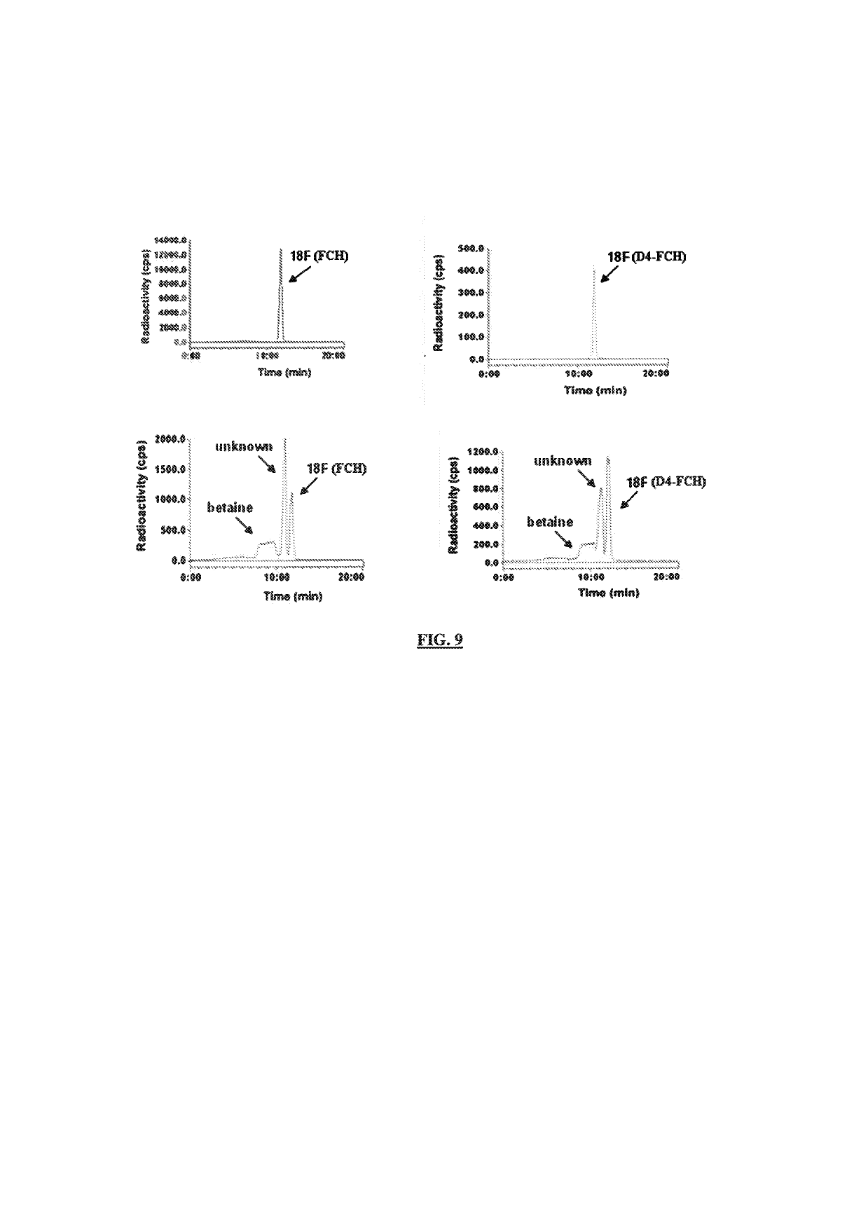

### Choline Oxidase Model

[¹⁸F]fluoromethylcholine and [¹⁸F]fluoromethyl-[1,2-²H₄]choline were evaluated in a choline oxidase model (Roivainen, A., et al., European Journal of Nuclear Medicine 2000; 27:25-32). The graphical representation in FIG. 8 clearly shows that, in the enzymatic oxidative model, the deuterated compound is significantly more stable than the corresponding non-deuterated compound. At the 60 minute time point the radio-HPLC distribution of choline species revealed that for [¹⁸F]fluoromethylcholine the parent radiotracer was present at the level of 11±8%; at 60 minutes the corresponding parent deuterated radiotracer [¹⁸F]fluoromethyl-[1,2-²H₄]choline was present at 29±4%. Relevant radio-HPLC chromatograms are shown in FIG. 9 and further exemplify the increased oxidative stability of [¹⁸F]fluoromethyl-[1,2-²H₄]-choline relative to [¹⁸F]fluoromethylcholine. These radio-HPLC chromatograms contain a third peak, marked as ‘unknown’, that is speculated to be the intermediate oxidation product, betaine aldehyde.

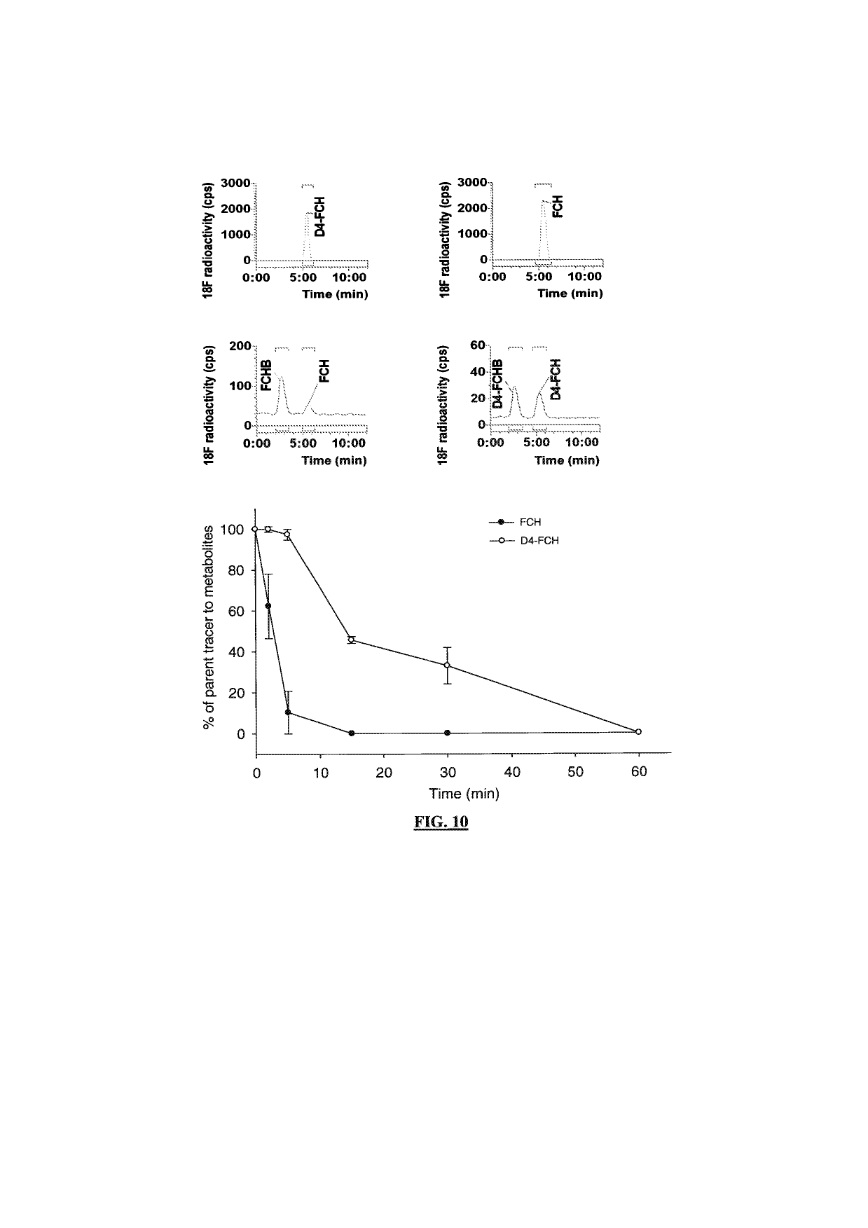

### In Vivo Stability Analysis

[¹⁸F]fluoromethyl-[1,2-²H₄]-choline is more resistant to oxidation in vivo. The relative rates of oxidation of the two isotopically radiolabeled choline species, [¹⁸F]fluoromethylcholine and [¹⁸F]fluoromethyl-[1,2-²H₄]-choline to their respective metabolites, [¹⁸F]fluoromethylcholine-betaine ([¹⁸F]-FCH-betaine) and [¹⁸F]fluoromethyl-[1,2-²H₄]-choline-betaine ([¹⁸F]D4-FCH-betaine) was evaluated by high performance liquid chromatography (HPLC) in mouse plasma after intravenous (i.v.) administration of the radiotracers. [¹⁸F]fluoromethyl-[1,2-²H₄]-choline was found to be markedly more stable to oxidation than [¹⁸F]fluoromethylcholine. As shown in FIG. 10, [¹⁸F]fluoromethyl-[1,2-²H₄]-choline was markedly more stable than [¹⁸F]fluoromethylcholine with 40% conversion of [¹⁸F]fluoromethyl-[1,2-²H₄]-choline to [¹⁸F]D4-FCH-betaine at 15 min after i.v. injection into mice compared to 80% conversion of [¹⁸F]fluoromethylcholine to [¹⁸F]-FCH-betaine. The time course for in vivo oxidation is shown in FIG. 10 showing overall improved stability of [¹⁸F]fluoromethyl-[1,2-²H₄]-choline over [¹⁸F]fluoromethylcholine.

### Biodistribution

### Time Course Biodistribution

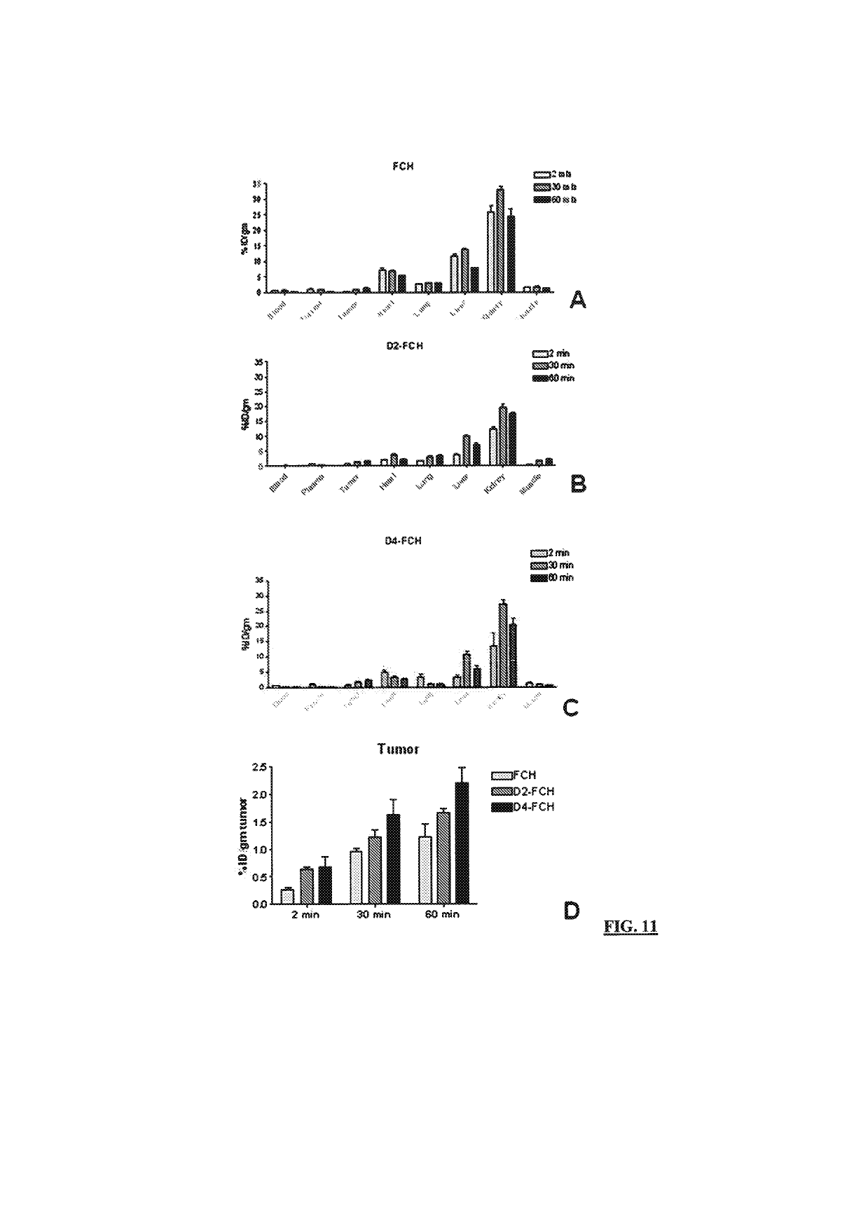

Time course biodistribution was carried out for [¹⁸F]fluoromethylcholine, [¹⁸F]fluoromethyl-[1-²H₂]choline and [¹⁸F]fluoromethyl-[1,2-²H₄]choline in nude mice bearing HCT116 human colon xenografts. Tissues were collected at 2, 30 and 60 minutes post-injection and the data summarized in FIG. 11A-C. The uptake values for [¹⁸F]fluoromethylcholine were in broad agreement with earlier studies (DeGrado, T. R., et al., “Synthesis and Evaluation of ¹⁸F-labeled Choline as an Oncologic Tracer for Positron Emisson Tomography: Initial Findings in Prostate Cancer”, Cancer Research 2000; 61:110-7). Comparison of the uptake profiles revealed a reduced uptake of radiotracer in the heart, lung and liver for the deuterated compounds [¹⁸F]fluoromethyl-[1-²H₂]-choline and [¹⁸F]fluoromethyl-[1,2-²H₄]-choline. The tumor uptake profile for the three radiotracers is shown in FIG. 11D and shows increased localization of radiotracer for the deuterated compounds relative to [¹⁸F]fluoromethylcholine at all time points. A pronounced increase in tumor uptake of [¹⁸F]fluoromethyl-[1,2-²H₄]choline at the later time points is evident.

### Distribution of Choline Metabolites

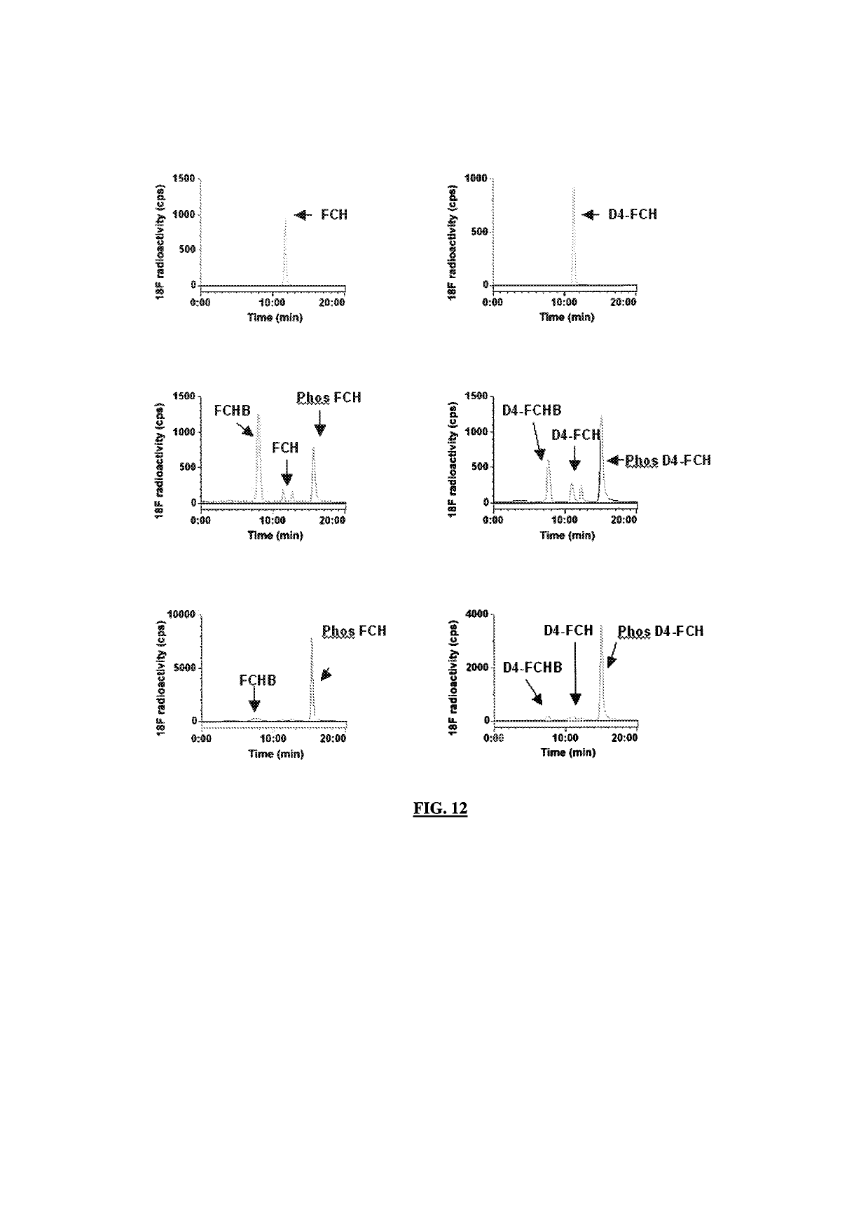

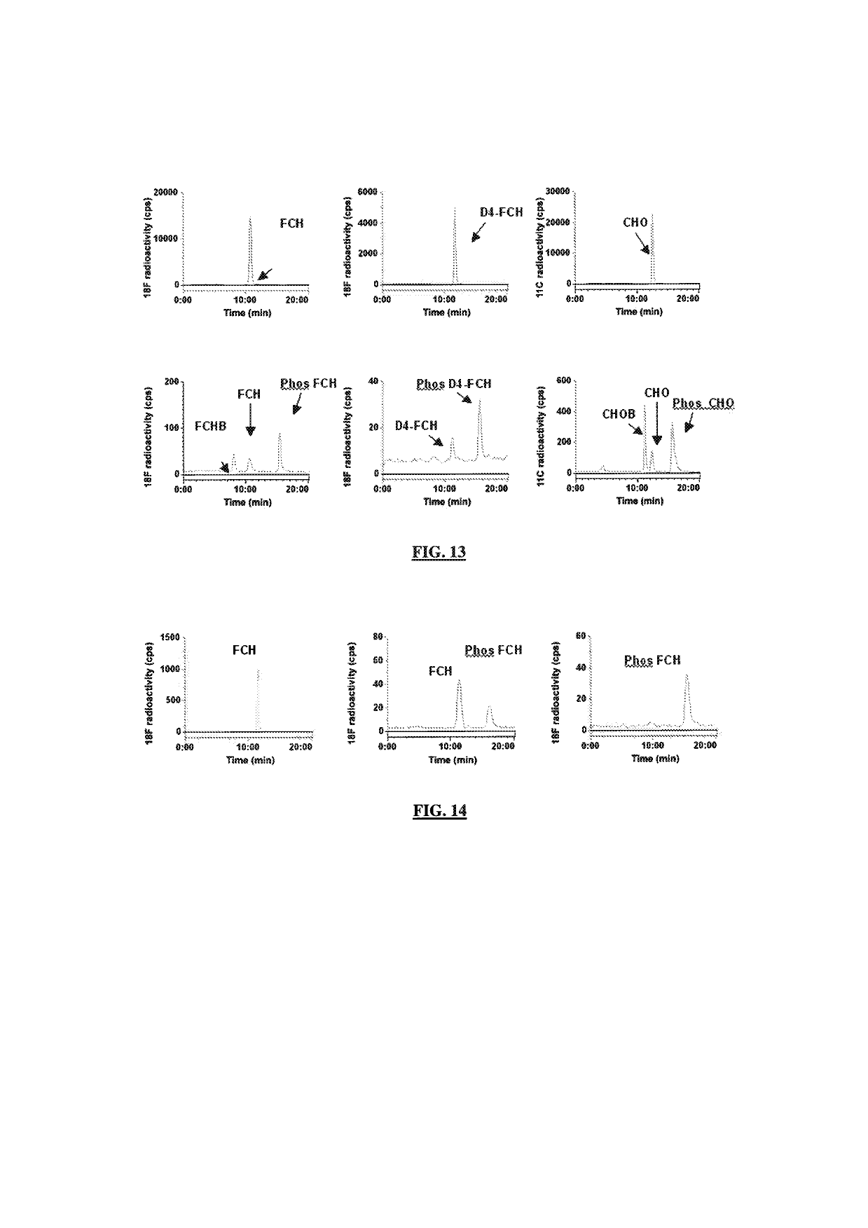

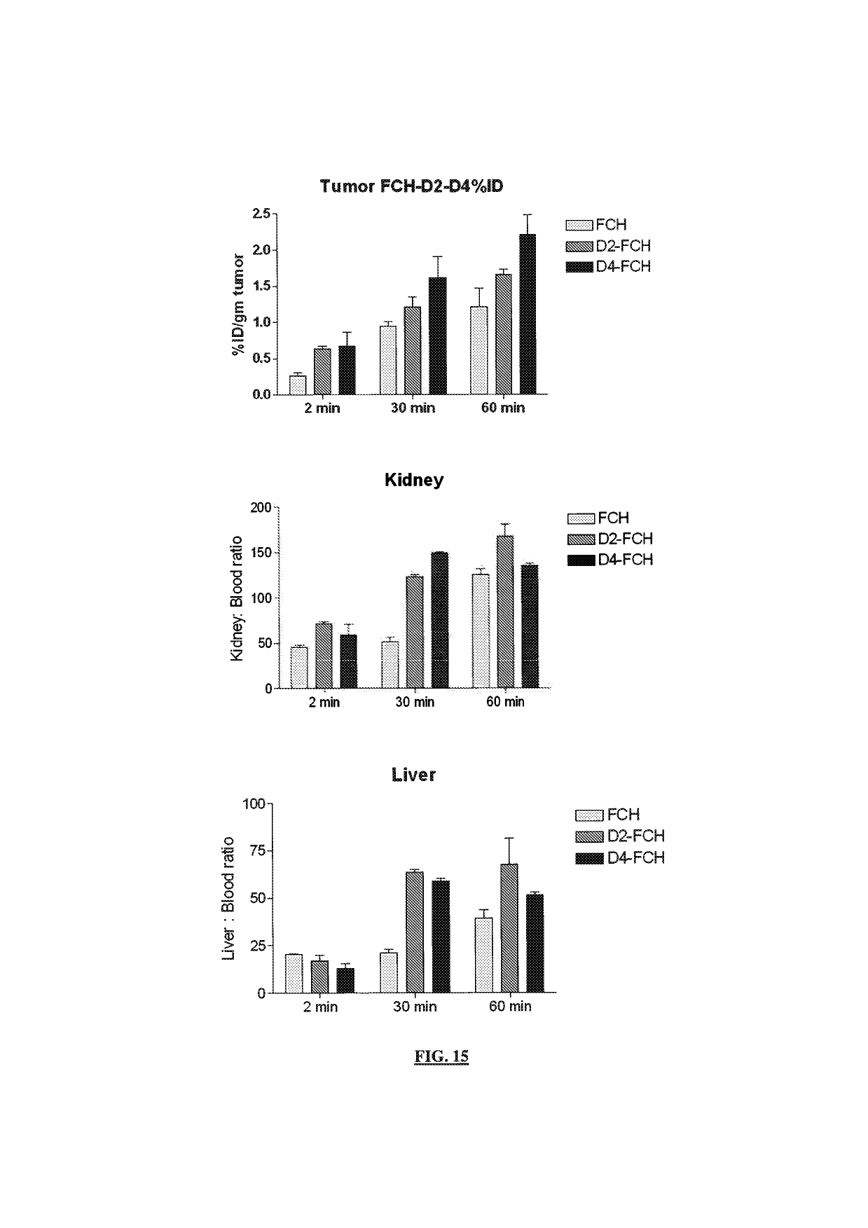

Metabolite analysis of tissues including liver, kidney and tumor by HPLC was also accomplished. Typical HPLC chromatograms of [¹⁸F]FCH and [¹⁸F]D4-FCH and their respective metabolites in tissues are shown in FIG. 12. Tumor distribution of metabolites was analyzed in a similar fashion (FIG. 13). Choline and its metabolites lack any UV chromophore to permit presentation of chromatograms of the cold unlabelled compound simultaneously with the radioactivity chromatograms. Thus, the presence of metabolites was validated by other chemical and biological means. Of note the same chromatographic conditions were used for characterization of the metabolites and retention times were similar. The identity of the phosphocholine peak was confirmed biochemically by incubation of the putative phosphocholine formed in untreated HCT116 tumor cells with alkaline phosphatase (FIG. 14). A high proportion of liver radioactivity was present as phosphocholine at 30 min post injection for both [¹⁸F]FCH and [¹⁸F]D4-FCH (FIG. 12). An unknown metabolite (possibly the aldehyde intermediate) was observed in both the liver (7.4±2.3%) and kidney (8.8±0.2%) samples of [¹⁸F]D4-FCH treated mice. In contrast, this unknown metabolite was not found in liver samples of [¹⁸F]FCH treated mice and only to a smaller extent (3.3±0.6%) in kidney samples. Notably 60.6±3.7% of [¹⁸F]D4-FCH derived kidney radioactivity was phosphocholine compared to 31.8±9.8% from [¹⁸F]FCH(P=0.03). Conversely, most of the [¹⁸F]FCH-derived radioactivity in the kidney was in the form of [¹⁸F]FCH-betaine; 53.5±5.3% compared to 20.6±6.2% for [¹⁸F]D4-FCH (FIG. 12). It could be argued that levels of betaine in plasma reflected levels in tissues such as liver and kidneys. Tumors showed a different HPLC profile compared to liver and kidneys; typical radio-HPLC chromatograms obtained from the analysis of tumor samples (30 min after intravenous injection of [¹⁸F]FCH, [¹⁸F]D4-FCH and [¹¹C]choline) are shown in FIG. 12. In tumors, radioactivity was mainly in the form of phosphocholine in the case of [¹⁸F]D4-FCH (FIG. 13). In contrast [¹⁸F]FCH showed significant levels of [¹⁸F]FCH-betaine. In the context of late imaging, these results indicate that [¹⁸F]D4-FCH will be the superior radiotracer for PET imaging with an uptake profile that is easier to interpret.

The suitable and preferred aspects of any feature present in multiple aspects of the present invention are as defined for said features in the first aspect in which they are described herein. The invention is now illustrated by a series of non-limiting examples.

### Isotopic Carbon Choline Analogs

The present invention provides a compound of Formula (III) as described herein. Such compounds are useful as PET imaging agents for tumor imaging, as described herein. In particular, a compound of Formula (III), as described herein, may not be excreted in the urine and hence provide more specific imaging of pelvic malignancies such as prostate cancer.

The present invention provides a method to prepare a compound for Formula (III), wherein said method comprises reaction of the precursor compound of Formula (II) with a compound of Formula (IV) to form a compound of Formula (III) (Scheme A):

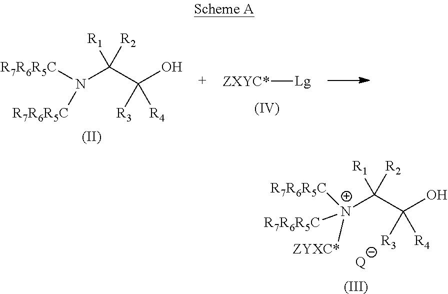

wherein the compounds of Formulae (I) and (III) are each as described herein and the compound of Formula (IV) is as follows:

ZXYC*-Lg (IV)

wherein C*, X, Y and Z are each as defined herein for a compound of Formula (III) and “Lg” is a leaving group. Suitable examples of “Lg” include, but are not limited to, bromine (Br) and tosylate (OTos). A compound of Formula (IV) can be prepared by any means known in the art including those described herein (e.g., analogous to Examples 5 and 7).

### EXAMPLES

Reagents and solvents were purchased from Sigma-Aldrich (Gillingham, UK) and used without further purification. Fluoromethylcholine chloride (reference standard) was purchased from ABCR Gmbh & Co. (Karlsruhe, Germany). Isotonic saline (0.9% w/v) was purchased from Hameln Pharmaceuticals (Gloucester, UK). NMR Spectra were obtained using either a Bruker Avance NMR machine operating at 400 MHz (¹H NMR) and 100 MHz (¹³C NMR) or 600 MHz (¹H NMR) and 150 MHz (¹³C NMR). Accurate mass spectroscopy was carried out on a Waters Micromass LCT Premier machine in positive electron ionisation (EI) or chemical ionisation (CI) mode. Distillation was carried out using a Büchi B-585 glass oven (Büchi, Switzerland).

### Example 1

### Preparation of N,N-dimethyl-[1,2-²H₄]-ethanolamine (3)

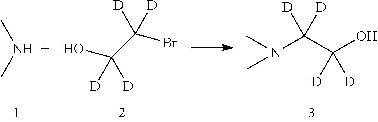

To a suspension of K₂CO₃ (10.50 g, 76 mmol) in dry THF (10 mL) was added dimethylamine (2.0 M in THF) (38 mL, 76 mmol) followed by 2-bromoethanol-1,1,2,2-d₄ (4.90 g, 38 mmol) and the suspension heated to 50° C. under argon. After 19 h, thin layer chromatography (TLC) (ethyl acetate/alumina/I₂) indicated complete conversion of (2) and the reaction mixture was allowed to cool to ambient temperature and filtered. Bulk solvent was then removed under reduced pressure. Distillation gave the desired product (3) as a colorless liquid, b.p. 78° C./88 mbar (1.93 g, 55%). ¹H NMR (CDCl₃, 400 MHz) δ 3.40 (s, 1H, OH), 2.24 (s, 6H, N(CH₃)₂). ¹³C NMR (CDCl₃, 75 MHz) δ 62.6 (NCD₂CD₂OH), 60.4 (NCD₂CD₂OH), 47.7 (N(CH₃)₂). HRMS (EI)=93.1093 (M⁺). C₄H₇²H₄N₀ requires 93.1092.

### Example 2

### Preparation of N,N-dimethyl-[1-²H₂]-ethanolamine (5)

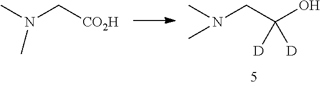

To a suspension of N,N-dimethylglycine (0.52 g, 5 mmol) in dry THF (10 mL) was added lithium aluminium deuteride (0.53 g, 12.5 mmol) and the resulting suspension refluxed under argon. After 24 h the suspension was allowed to cool to ambient temperature and poured onto sat. aq. Na₂SO₄ (15 mL) and adjusted to pH 8 with 1 M Na₂CO₃, then washed with ether (3×10 mL) and dried (Na₂SO₄). Distillation gave the desired product (5) as a colorless liquid, b.p. 65° C./26 mbar (0.06 g, 13%). ¹H NMR (CDCl₃, 400 MHz) δ 2.43 (s, 2H, NCH₂CD₂), 2.25 (s, 6H, N(CH₃)₂), 1.43 (s, 1H, OH). ¹³C NMR (CDCl₃, 150 MHz) δ 63.7 (NCH₂CD₂OH), 57.8 (NCH₂CD₂OH), 45.7 (N(CH₃)₂)

### Example 3

### Preparation of Fluoromethyltosylate (8)

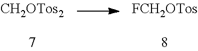

Methylene ditosylate (7) was prepared according to an established literature procedure and analytical data was consistent with reported values (Emmons, W. D., et al., Journal of the American Chemical Society, 1953; 75:2257; and Neal, T. R., et al., Journal of Labelled Compounds and Radiopharmaceuticals 2005; 48:557-68). To a solution of methylene ditosylate (7) (0.67 g, 1.89 mmol) in dry acetonitrile (10 mL) was added Kryptofix K_{222 [4,7,13,16,21,24}-hexaoxa-1,10-diazabicyclo[8.8.8]hexacosane] (1.00 g, 2.65 mmol) followed by potassium fluoride (0.16 g, 2.83 mmol). The suspension was then heated to 110° C. under nitrogen. After 1 h TLC (7:3 hexane/ethyl acetate/silica/UV₂₅₄) indicated complete conversion of (7). The reaction mixture was diluted with ethyl acetate (25 mL), washed with water (2×15 mL) and dried over MgSO₄. Chromatography (5→10% ethyl acetate/hexane) gave the desired product (8) as a colorless oil (40 mg, 11%). ¹H NMR (CDCl₃, 400 MHz) δ 7.86 (d, 2H, J=8 Hz, aryl CH), 7.39 (d, 2H, J=8 Hz, aryl CH), 5.77 (d, 1H, J=52 Hz, CH₂F), 2.49 (s, 3H, tolyl CH₃). ¹³C NMR (CDCl₃) δ 145.6 (aryl), 133.8 (aryl), 129.9 (aryl), 127.9 (aryl), 98.1 (d, J=229 Hz, CH₂F), 21.7 (tolyl CH₃). HRMS (CI)=222.0604 (M+NH₄)⁺. Calcd. for C₈H₁₃FNO₃S 222.0600.

### Example 4

### Preparation of N,N-Dimethylethanolamine(O-4-methoxybenzyl) ether (O-PMB-DMEA)

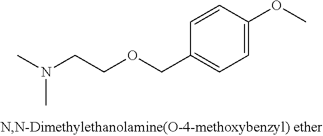

To a dry flask was added dimethylethanolamine (4.46 g, 50 mmol) and dry DMF (50 mL). The solution was stirred under argon and cooled in an ice bath. Sodium hydride (2.0 g, 50 mmol) was then added portionwise over 10 min and the reaction mixture then allowed to warm to room temperature. After 30 min 4-methoxybenzyl chloride (3.92 g, 25 mmol) was added dropwise over 10 min and the resulting mixture left to stir under argon. After 60 h GC-MS indicated reaction completion (disappearance of 4-methoxybenzyl chloride) and the reaction mixture was poured onto 1M sodium hydroxide (100 mL) and extracted with dichloromethane (DCM)(3×30 mL) then dried (Na₂SO₄). Column chromatography (0>10% methanol/DCM; neutral silica) gave the desired product (O-PMB-DMEA) as a yellow oil (1.46 g, 28%). ¹H NMR (CDCl₃, 400 MHz) δ 7.28 (d, 2H, J=8.6 Hz, aryl CH), 6.89 (d, 2H, J=8.6 Hz, aryl CH), 4.49 (s, 2H, —CH₂—), 3.81 (s, 3H, OCH₃), 3.54 (t, 2H, J=5.8, NCH₂CH₂O), 2.54 (t, 2H, J=5.8, NCH₂CH₂O), 2.28 (s, 6H, N(CH₃)₂). HRMS (ES)=210.1497 (M+H⁺). C₁₂H₂₀NO₂ requires 210.1494.

### Example 4a

### Preparation of Dueterated Analogues of N,N-Dimethylethanolamine(O-4-methoxybenzyl)ether (O-PMB-DMEA)

The di- and tetra-deuterated analogs of N,N-Dimethylethanolamine(O-4-methoxybenzyl)ether can be prepared according to Example 4 from the appropriate di- or tetra-deuterated dimethylethanolamine.

### Example 5

### Preparation of Synthesis of [¹⁸F]fluoromethyl tosylate (9)

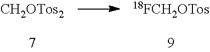

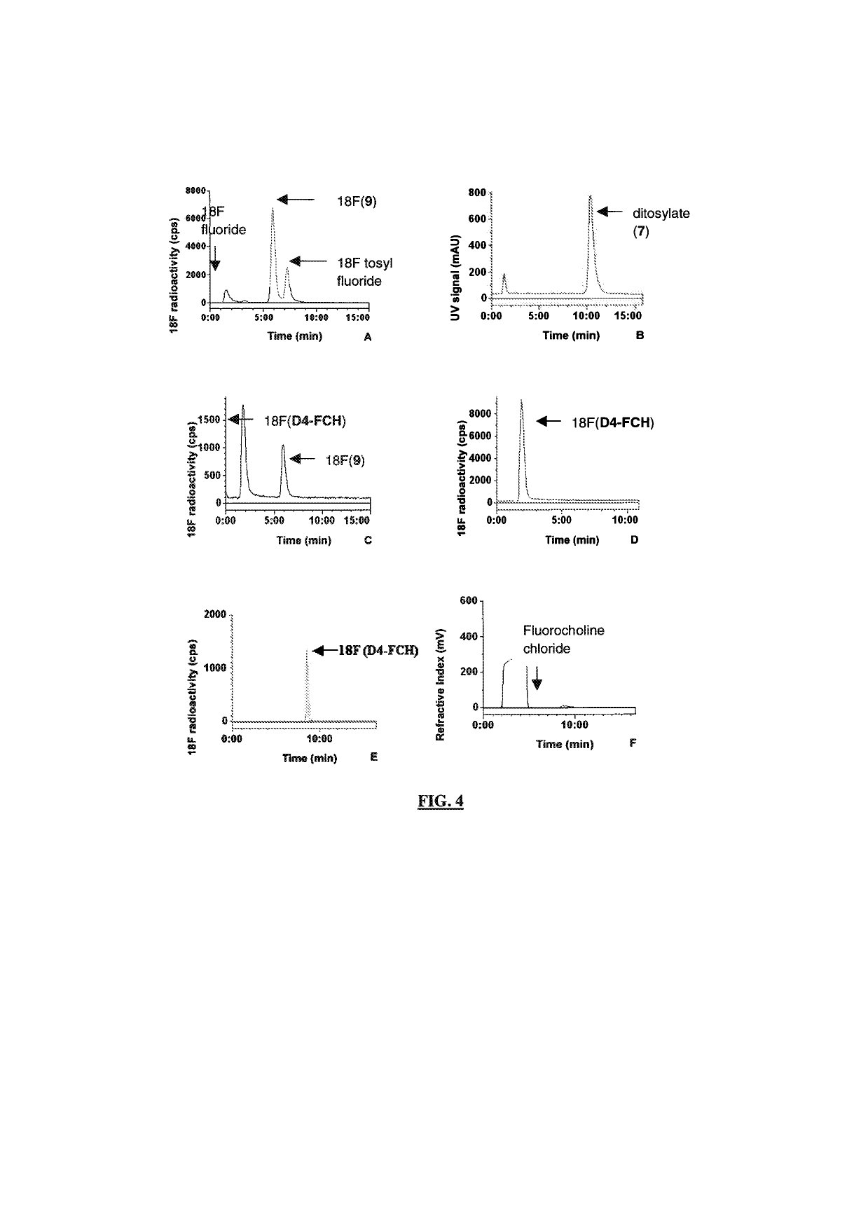

To a Wheaton vial containing a mixture of K₂CO₃ (0.5 mg, 3.6 μmol, dissolved in 100 μL water), 18-crown-6 (10.3 mg, 39 μmol) and acetonitrile (500 μL) was added [¹⁸F]fluoride (˜20 mCi in 100 μL water). The solvent was then removed at 110° C. under a stream of nitrogen (100 mL/min). Afterwards, acetonitrile (500 μL) was added and distillation to dryness continued. This procedure was repeated twice. A solution of methylene ditosylate (7) (6.4 mg, 18 μmol) in acetonitrile (250 μL) containing 3% water was then added at ambient temperature followed by heating at 100° C. for 10-15 min, with monitoring by analytical radio-HPLC. The reaction was quenched by addition of 1:1 acetonitrile/water (1.3 mL) and purified by semi-preparative radio-HPLC. The fraction of eluent containing [¹⁸F]fluoromethyl tosylate (9) was collected and diluted to a final volume of 20 mL with water, then immobilized on a Sep Pak C18 light cartridge (Waters, Milford, Mass., USA) (pre-conditioned with DMF (5 mL) and water (10 mL)). The cartridge was washed with further water (5 mL) and then the cartridge, with [¹⁸F]fluoromethyl tosylate (9) retained, was dried in a stream of nitrogen for 20 min. A typical HPLC reaction profile for synthesis of [¹⁸F](13) is shown in FIG. 4A/4B below.

### Example 6

### Radiosynthesis of [¹⁸F]Fluoromethylcholine Derivatives by Reaction with [¹⁸F]fluorobromomethane

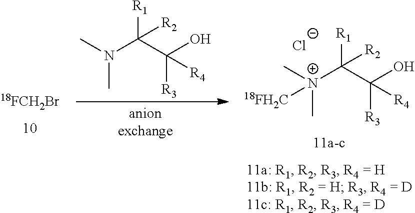

[¹⁸F]Fluorobromomethane (prepared according to Bergman et al (Appl Radiat Isot 2001; 54(6):927-33)) was added to a Wheaton vial containing the amine precursor N,N-dimethylethanolamine (150 μL) or N,N-dimethyl-[1,2-²H₄]ethanolamine (3) (150 μL) in dry acetonitrile (1 mL), pre-cooled to 0° C. The vial was sealed and then heated to 100° C. for 10 min Bulk solvent was then removed under a stream of nitrogen, then the sample remaining was redissolved in 5% ethanol in water (10 mL) and immobilized on a Sep-Pak CM light cartridge (Waters, Milford, Mass., USA) (pre-conditioned with 2 M HCl (5 mL) and water (10 mL)) to effect the chloride anion exchange. The cartridge was then washed with ethanol (10 mL) and water (10 mL) followed by elution of the radiotracer (11a) or (11c) using saline (0.5-2.0 mL) and passing through a sterile filter (0.2 μm) (Sartorius, Goettingen, Germany).

### Example 7

### Radiosynthesis of [¹⁸F]Fluoromethylcholine, [¹⁸F]fluoromethyl-[1-²H₂]choline and [¹⁸F]fluoromethyl-[1,2-²H₄]choline by reaction with [¹⁸F]fluoromethylmethyl tosylate

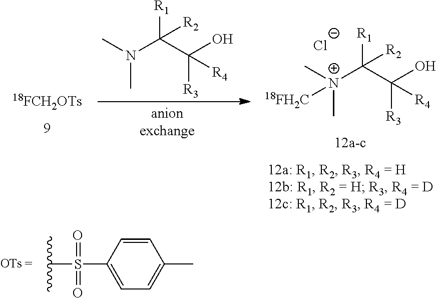

[¹⁸F]Fluoromethyl tosylate (9) (prepared according to Example 5) and eluted from the Sep-Pak cartridge using dry DMF (300 pt), was added in to a Wheaton vial containing one of the following precursors: N,N-dimethylethanolamine (150 pt); N,N-dimethyl-[1,2-²H₄]ethanolamine (3) (150 pt) (prepared according to Example 1); or N,N-dimethyl-[1-²H₂]ethanolamine (5) (150 pt) (prepared according to Example 2), and heated to 100° C. with stiffing. After 20 min the reaction was quenched with water (10 mL) and immobilized on a Sep Pak CM light cartridge (Waters) (pre-conditioned with 2M HCl (5 mL) and water (10 mL)) in order to effect the chloride anion exchange and then washed with ethanol (5 mL) and water (10 mL) followed by elution of the radiotracer [¹⁸F]Fluoromethylcholine (12a), [¹⁸F]fluoromethyl-[1-²H₂]choline (12b) or [¹⁸F]fluoromethyl-[1,2-²H₄]choline [¹⁸F] (12c) with isotonic saline (0.5-1.0 mL).

### Example 8

### Synthesis of cold Fluoromethyltosylate (15)

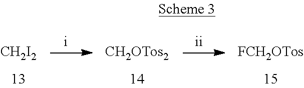 [0224] i: Silver p-toluenesulfonate, MeCN, reflux, 20 h;
ii: KF, MeCN, reflux, 1 h.
   According to Scheme 3 above:
   (a) Synthesis of methylene ditosylate (14)

Commercially available diiodomethane (13) (2.67 g, 10 mmol) was reacted with silver tosylate (6.14 g, 22 mmol), using the method of Emmons and Ferris, to give methylene ditosylate (10) (0.99 g) in 28% yield (Emmons, W. D., et al., “Metathetical Reactions of Silver Salts in Solution. II. The Synthesis of Alkyl Sulfonates”, Journal of the American Chemical Society, 1953; 75:225).

(b) Synthesis of cold Fluoromethyltosylate (15)
Fluoromethyltosylate (11) (0.04 g) was prepared by nucleophilic substitution of Methylene ditosylate (10) (0.67 g, 1.89 mmol) of Example 3(a) using potassium fluoride (0.16 g, 2.83 mmol)/Kryptofix K₂₂₂ (1.0 g, 2.65 mmol) in acetonitrile (10 mL) at 80° C. to give the desired product in 11% yield.

### Example 9

### Synthesis of [¹⁸F]fluorobromomethane (17)

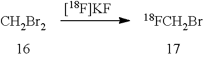

Adapting the method of Bergman et al (Appl Radiat Isot 2001; 54(6):927-33), commercially available dibromomethane (16) is reacted with [¹⁸F]potassium fluoride/Kryptofix K₂₂₂ in acetonitrile at 110° C. to give the desired [¹⁸F]fluorobromomethane (17), which is purified by gas-chromatography and trapped by elution into a pre-cooled vial containing acetonitrile and the relevant choline precursor.

### Example 10

### Analysis of Radiochemical Purity

Radiochemical purity for [¹⁸F]Fluoromethylcholine, [¹⁸F]fluoromethyl-[1-²H₂]choline and [¹⁸F]fluoromethyl-[1,2-²H₄]choline [¹⁸F] was confirmed by co-elution with a commercially available fluorocholine chloride standard. An Agilent 1100 series HPLC system equipped with an Agilent G1362A refractive index detector (RID) and a Bioscan Flowcount FC-3400 PIN diode detector was used. Chromatographic separation was performed on a Phenomenex Luna C₁₈ reverse phase column (150 mm×4.6 mm) and a mobile phase comprising of 5 mM heptanesulfonic acid and acetonitrile (90:10 v/v) delivered at a flow rate of 1.0 mL/min

### Example 11

### Enzymatic Oxidation Study Using Choline Oxidase

This method was adapted from that of Roivannen et al (Roivainen, A., et al., European Journal of Nuclear Medicine 2000; 27:25-32). An aliquot of either [¹⁸F]Fluoromethylcholine or [¹⁸F]fluoromethyl-[1,2-²H₄]choline [¹⁸F] (100 μL, ˜3.7 MBq) was added to a vial containing water (1.9 mL) to give a stock solution. Sodium phosphate buffer (0.1 M, pH 7) (10 uL) containing choline oxidase (0.05 units/uL) was added to an aliquot of stock solution (190 uL) and the vial was then left to stand at room temperature, with occasional agitation. At selected time-points (5, 20, 40 and 60 minutes) the sample was diluted with HPLC mobile phase (buffer A, 1.1 mL), filtered (0.22 μm filter) and then 1 mL injected via a 1 mL sample loop onto the HPLC for analysis. Chromatographic separation was performed on a Waters C₁₈ Bondapak (7.8×300 mm) column (Waters, Milford, Mass., USA) at 3 mL/min with a mobile phase of buffer A, which contained acetonitrile, ethanol, acetic acid, 1.0 mol/L ammonium acetate, water, and 0.1 mol/L sodium phosphate (800:68:2:3:127:10 [v/v]) and buffer B, which contained the same constituents but in different proportions (400:68:44:88:400:10 [v/v]). The gradient program comprised 100% buffer A for 6 minutes, 0-100% buffer B for 10 minutes, 100-0% B in 2 minutes then 0% B for 2 minutes.

### Example 12

### Biodistribution

Human colon (HCT116) tumors were grown in male C3H-Hej mice (Harlan, Bicester, United Kingdom) as previously reported (Leyton, J., et al., Cancer Research 2005; 65(10):4202-10). Tumor dimensions were measured continuously using a caliper and tumor volumes were calculated by the equation: volume=(π/6)×a×b×c, where a, b, and c represent three orthogonal axes of the tumor. Mice were used when their tumors reached approximately 100 mm³. [¹⁸F]Fluoromethylcholine, [¹⁸F]fluoromethyl-[1-²H₂]choline and [¹⁸F]fluoromethyl-[1,2-²H₄]choline (˜3.7 MBq) were each injected via the tail vein into awake untreated tumor bearing mice. The mice were sacrificed at pre-determined time points (2, 30 and 60 min) after radiotracer injection under terminal anesthesia to obtain blood, plasma, tumor, heart, lung, liver, kidney and muscle. Tissue radioactivity was determined on a gamma counter (Cobra II Auto-Gamma counter, Packard Biosciences Co, Pangbourne, UK) and decay corrected. Data were expressed as percent injected dose per gram of tissue.

### Example 13

### Oxidation Potential of [¹⁸F]Fluoromethylcholine ([¹⁸F]FCH) and [¹⁸F]fluoromethyl-[1,2-²H₄]choline ([¹⁸F]D4-FCH) in vivo

[¹⁸F]FCH or [¹⁸F](D4-FCH) (80-100 μCi) was injected via the tail vein into anesthetized non-tumor bearing C3H-Hej mice; isofluorane/O₂/N₂O anesthesia was used. Plasma samples obtained at 2, 15, 30 and 60 minutes after injection were snap frozen in liquid nitrogen and stored at −80° C. For analysis, samples were thawed and kept at 4° C. To approximately 0.2 mL of plasma was added ice-cold acetonitrile (1.5 mL). The mixture was then centrifuged (3 minutes, 15,493×g; 4° C.). The supernatant was evaporated to dryness using a rotary evaporator (Heidoloph Instruments GMBH & CO, Schwabach, Germany) at a bath temperature of 45° C. The residue was suspended in mobile phase (1.1 mL), clarified (0.2 μm filter) and analyzed by HPLC. Liver samples were homogenized in ice-cold acetonitrile (1.5 mL) and then subsequently treated as per plasma samples. All samples were analyzed on an Agilent 1100 series HPLC system equipped with a γ-RAM Model 3 radio-detector (IN/US Systems inc., FL, USA). The analysis was based on the method of Roivannen (Roivainen, A., et al., European Journal of Nuclear Medicine 2000; 27:25-32) using a Phenomenex Luna SCX column (10μ, 250×4 6 mm) and a mobile phase comprising of 0.25 M sodium dihydrogen phosphate (pH 4.8) and acetonitrile (90:10 v/v) delivered at a flow rate of 2 ml/min.

### Example 14

### Distribution of Choline Metabolites

Liver, kidney, and tumor samples were obtained at 30 min. All samples were snap-frozen in liquid nitrogen. For analysis, samples were thawed and kept at 4° C. immediately before use. To ˜0.2 mL plasma was added ice-cold methanol (1.5 mL). The mixture was then centrifuged (3 min, 15,493×g, 4jC). The supernatant was evaporated to dryness using a rotary evaporator (Heidoloph Instruments) at a bath temperature of 40° C. The residue was suspended in mobile phase (1.1 mL), clarified (0.2 Am filter), and analyzed by HPLC. Liver, kidney, and tumor samples were homogenized in ice-cold methanol (1.5 mL) using an IKA Ultra-Turrax T-25 homogenizer and subsequently treated as per plasma samples (above). All samples were analyzed by radio-HPLC on an Agilent 1100 series HPLC system (Agilent Technologies) equipped with a γ-RAM Model 3 γ-detector (IN/US Systems) and Laura 3 software (Lablogic). The stationary phase comprised a Waters μBondapak C18 reverse-phase column (300×7.8 mm) (Waters, Milford, Mass., USA). Samples were analyzed using a mobile phase comprising solvent A (acetonitrile/water/ethanol/acetic acid/1.0 mol/L ammonium acetate/0.1 mol/L sodium phosphate; 800/127/68/2/3/10) and solvent B (acetonitrile/water/ethanol/acetic acid/1.0 mol/L ammonium acetate/0.1 mol/L sodiumphosphate; 400/400/68/44/88/10) with a gradient of 0% B for 6 min, then 0>100% B in 10 min, 100% B for 0.5 min, 100>0% B in 1.5 min then 0% B for 2 min, delivered at a flow rate of 3 mL/min

### Example 15

### Metabolism of [¹⁸F]D4-FCH and [¹⁸F]FCH by HCT116 Tumor Cells

HCT116 cells were grown in T150 flasks in triplicate until they were 70% confluent and then treated with vehicle (1% DMSO in growth medium) or 1 μmol/L PD0325901 in vehicle for 24 h. Cells were pulsed for 1 h with 1.1 MBq of either [¹⁸F]D4-FCH or [¹⁸F]FCH. The cells were washed three times in ice-cold phosphate buffered saline (PBS), scraped into 5 mL PBS, and centrifuged at 500×g for 3 min and then resuspended in 2 mL ice-cold methanol for HPLC analysis as described above for tissue samples. To provide biochemical evidence that the 5′-phosphate was the peak identified on the HPLC chromatogram, cultured cells were treated with alkaline phosphatase as described previously (Barthel, H., et al., Cancer Res 2003; 63(13):3791-8). Briefly, HCT116 cells were grown in 100 mm dishes in triplicate and incubated with 5.0 MBq [¹⁸F]FCH for 60 min at 37° C. to form the putative [¹⁸F]FCH-phosphate. The cells were washed with 5 mL ice-cold PBS twice and then scraped and centrifuged at 750×g (4° C., 3 min) in 5 mL PBS. Cells were homogenized in 1 mL of 5 mmol/L Tris-HCl (pH 7.4) containing 50% (v/v) glycerol, 0.5 mmol/L MgCl₂, and 0.5 mmol/L ZnCl₂ and incubated with 10 units bacterial (type III) alkaline phosphatase (Sigma) at 37° C. in a shaking water bath for 30 min to dephosphorylate the [¹⁸F]FCH-phosphate. The reaction was terminated by adding ice-cold methanol. Samples were processed as per plasma above and analyzed by radio-HPLC. Control experiments were done without alkaline phosphatase.

### Example 16

### Small Animal PET Imaging

### PET Imaging Studies.

Dynamic [¹⁸F]FCH and [¹⁸F]D4-FCH imaging scans were carried out on a dedicated small animal PET scanner, quad-HIDAC (Oxford Positron Systems). The features of this instrument have been described previously (Barthel, H., et al., Cancer Res 2003; 63(13):3791-8). For scanning the tail veins, vehicle- or drug-treated mice were cannulated after induction of anesthesia (isofluorane/O₂/N₂O). The animals were placed within a thermostatically controlled jig (calibrated to provide a rectal temperature of ˜37° C.) and positioned prone in the scanner. [¹⁸F]FCH or [¹⁸F]D4-FCH (2.96-3.7 MBq) was injected via the tail vein cannula and scanning commenced. Dynamic scans were acquired in list mode format over a 60 min period as reported previously (Leyton, J., et al., Cancer Research 2006; 66(15):7621-9). The acquired data were sorted into 0.5 mm sinogram bins and 19 time frames (0.5×0.5×0.5 mm voxels; 4×15, 4×60, and 11×300 s) for image reconstruction, which was done by filtered back-projection using a two-dimensional Hamming filter (cutoff 0.6). The image data sets were visualized using the Analyze software (version 6.0; Biomedical Imaging Resource, Mayo Clinic). Cumulative images of 30 to 60 min dynamic data were used for visualization of radiotracer uptake and to draw regions of interest. Regions of interest were defined manually on five adjacent tumor regions (each 0.5 mm thickness). Dynamic data from these slices were averaged for each tissue (liver, kidney, muscle, urine, and tumor) and at each of the 19 time points to obtain time versus radioactivity curves. Corresponding whole body time versus radioactivity curves representing injected radioactivity were obtained by adding together radioactivity in all 200×160×160 reconstructed voxels. Tumor radioactivity was normalized to whole-body radioactivity and expressed as percent injected dose per voxel (% ID/vox). The normalized uptake of radiotracer at 60 min (% ID/vox60) was used for subsequent comparisons. The average of the normalized maximum voxel intensity across five slices of tumor % IDvox60max was also use for comparison to account for tumor heterogeneity and existence of necrotic regions in tumor. The area under the curve was calculated as the integral of % ID/vox from 0 to 60 min

### Example 17

### Effect of PD0325901 Treatment in Mice

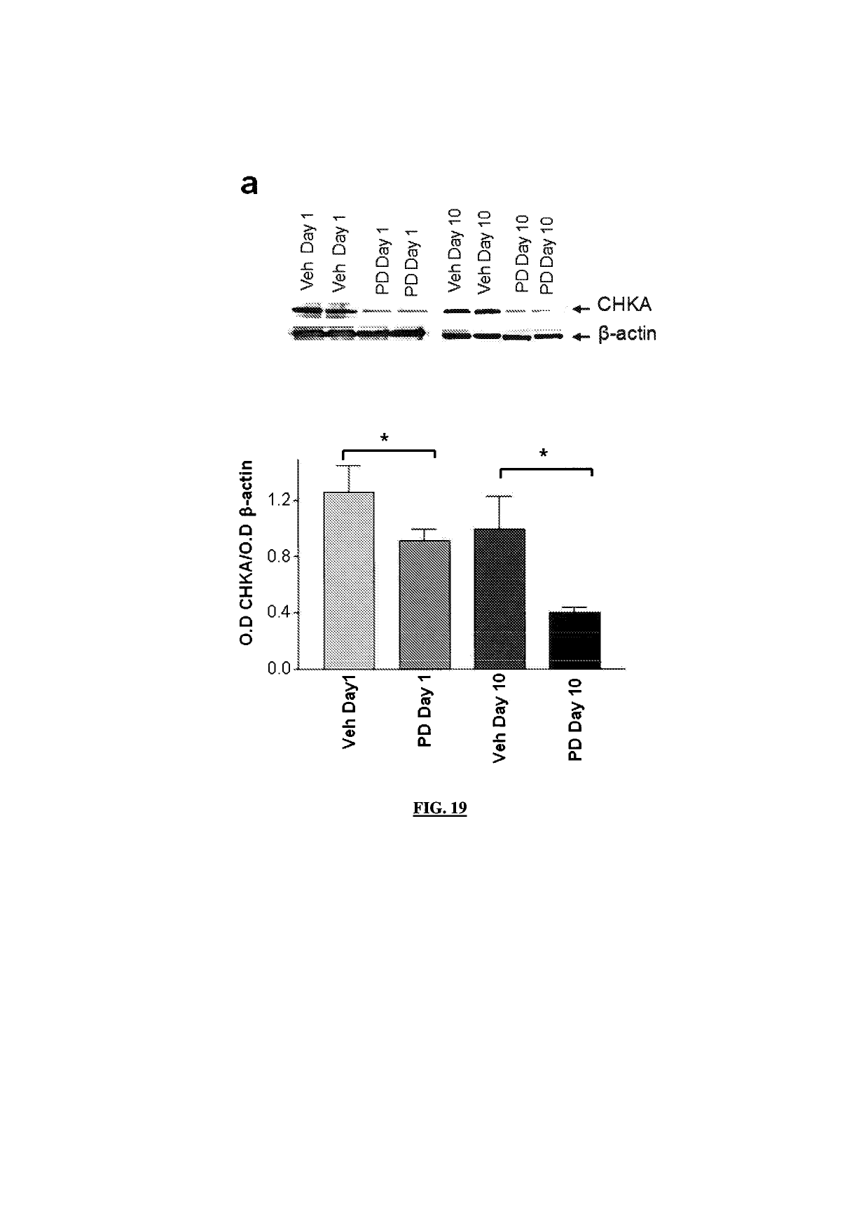

Size-matched HCT116 tumor bearing mice were randomized to receive daily treatment by oral gavage of vehicle (0.5% hydroxypropyl methylcellulose+0.2% Tween 80) or 25 mg/kg (0.005 mL/g mouse) of the mitogenic extracellular kinase inhibitor, PD0325901, prepared in vehicle. [¹⁸F]D4-FCH-PET scanning was done after 10 daily treatments with the last dose administered 1 h before scanning. After imaging, tumors were snap-frozen in liquid nitrogen and stored at ˜80° C. for analysis of choline kinase A expression. The results are illustrated in FIGS. 18 and 19.

This exemplifies use of [¹⁸F]D4-FCH-PET as an early biomarker of drug response. Most of the current drugs in development for cancer target key kinases involved in cell proliferation or survival. This example shows that in a xenograft model for which tumor shrinkage is not significant, growth factor receptor-Ras-MAP kinase pathway inhibition by the MEK inhibitor PD0325901 leads to a significant reduction in tumor [¹⁸F]D4-FCH uptake signifying inhibition of the pathway. The figure also shows that inhibition of [¹⁸F]D4-FCH uptake was due at least in part to the inhibition of choline kinase activity.

### Example 18

### Comparison of [¹⁸F]FCH and [¹⁸F]D4-FCH for Imaging

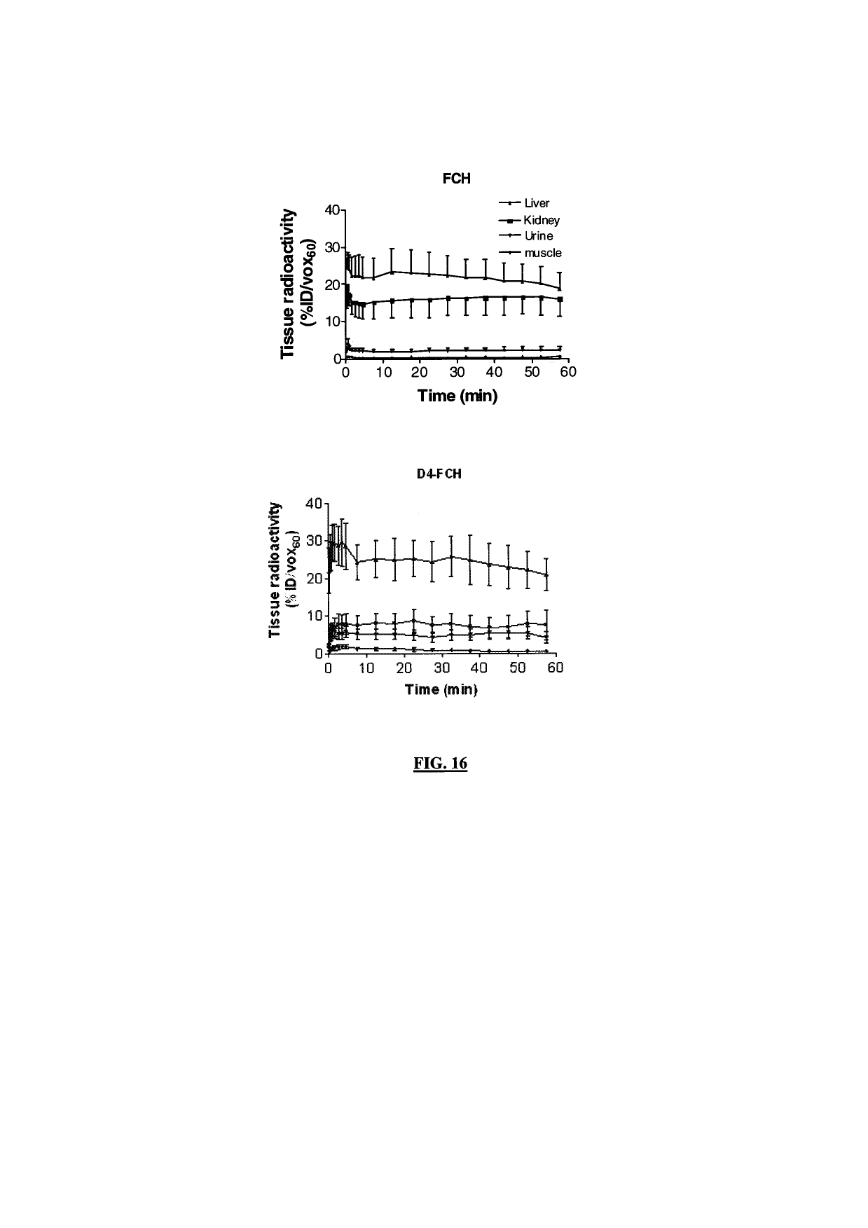

As illustrated in FIG. 16, [¹⁸F]FCH and [¹⁸F]D4-FCH were both rapidly taken up into tissues and retained. Tissue radioactivity increased in the following order: muscle <urine <kidney <liver. Given the predominance of phosphorylation over oxidation in the liver (FIG. 12), little differences were found in overall liver radioactivity levels between the two radiotracers. Liver radioactivity at levels 60 min after [¹⁸F]D4-FCH or [¹⁸F]FCH injection, % ID/vox₆₀, was 20.92±4.24 and 18.75±4.28, respectively (FIG. 16). This is also in keeping with the lower levels betaine with [¹⁸F]D4-FCH injection than with [¹⁸F]FCH injection (FIG. 12). Thus, pharmacokinetics of the two radiotracers in liver determined by PET (which lacks chemical resolution) were similar. The lower kidney radioactivity levels for [¹⁸F]D4-FCH compared to [¹⁸F]FCH (FIG. 16), on the other hand, reflect the lower oxidation potential of [¹⁸F]D4-FCH in kidneys. The % ID/vox₆₀ for [¹⁸F]FCH and [¹⁸F]D4-FCH were 15.97±4.65 and 7.59±3.91, respectively in kidneys (FIG. 16). Urinary excretion was similar between the radiotracers. Regions of interest (ROIs) that were drawn over the bladder showed % ID/vox₆₀ values of 5.20±1.71 and 6.70±0.71 for [¹⁸F]D4-FCH and [¹⁸F]FCH, respectively. Urinary metabolites comprised mainly of the unmetabolized radiotracers. Muscle showed the lowest radiotracer levels of any tissue.

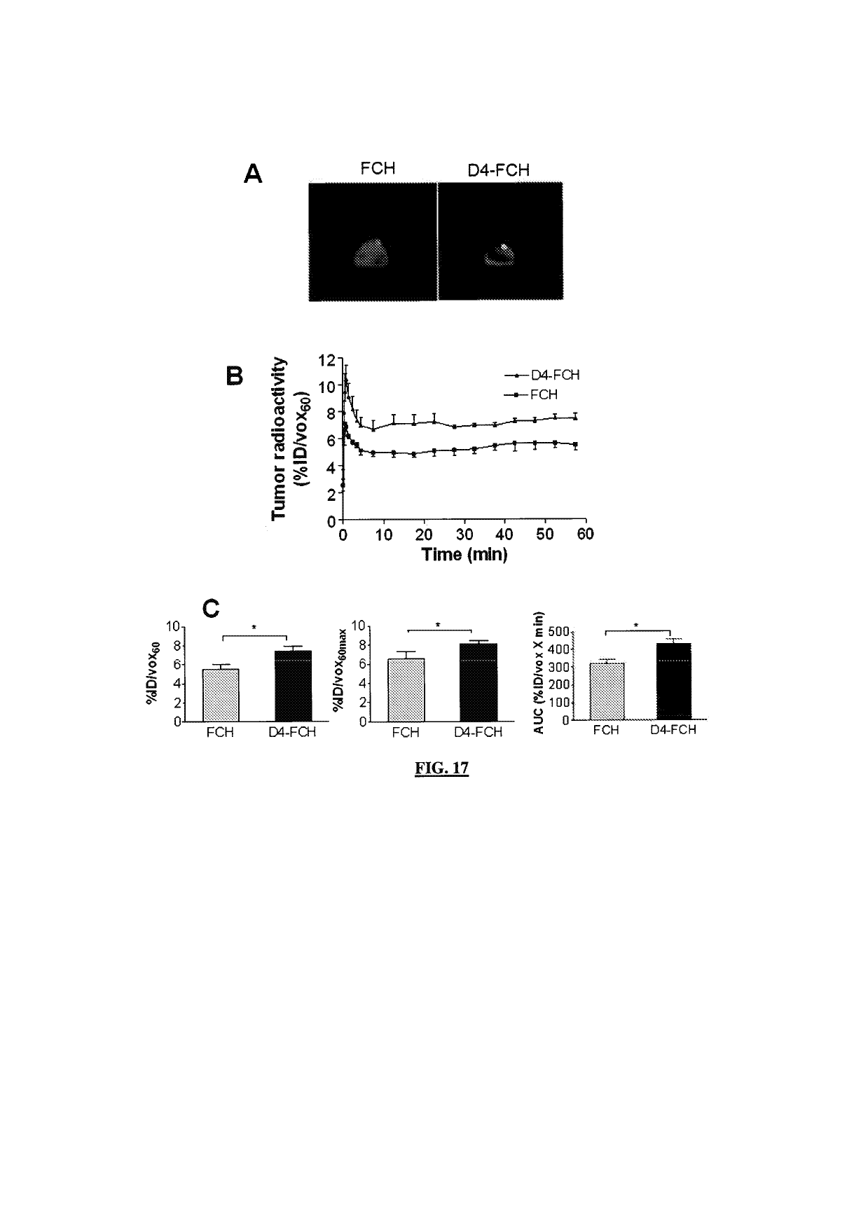

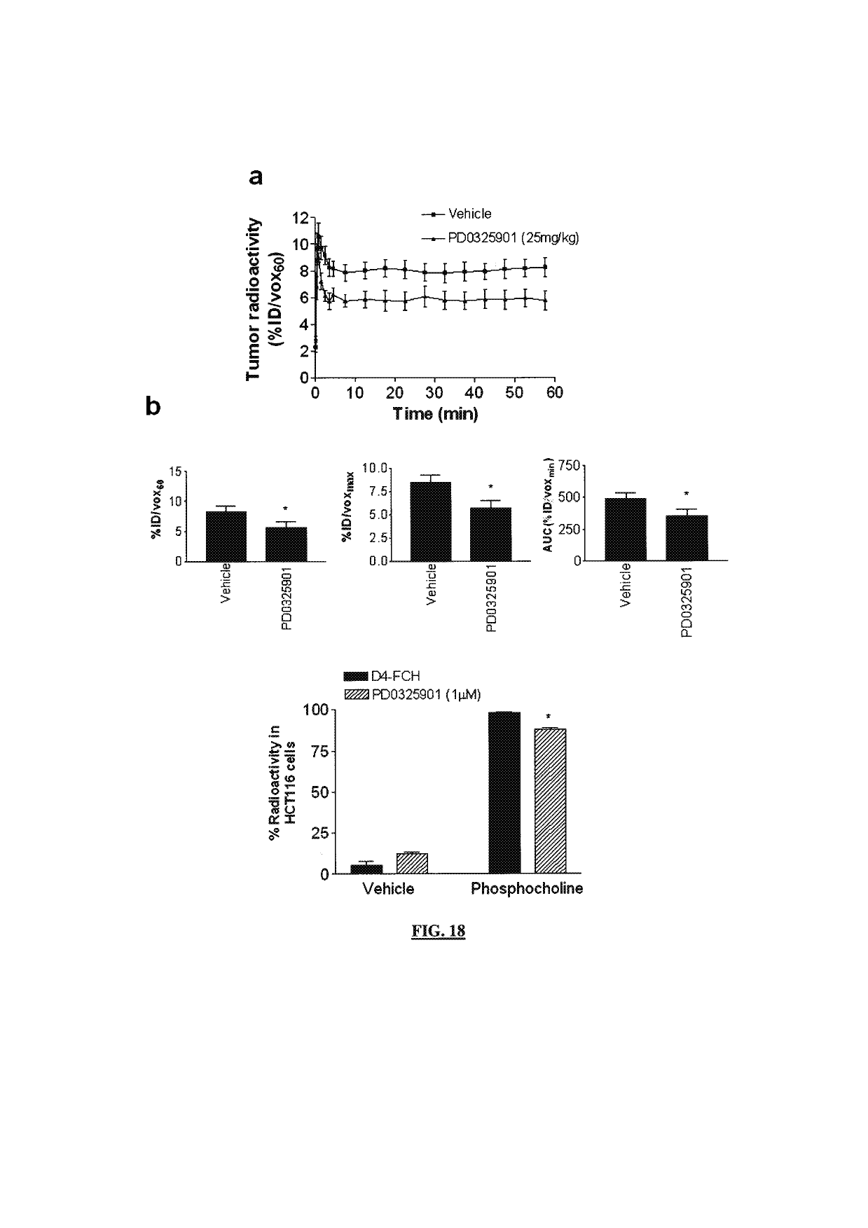

Despite the relatively high systemic stability of [¹⁸F]D4-FCH and high proportion of phosphocholine metabolites, higher tumor radiotracer uptake by PET in mice that were injected with [¹⁸F]D4-FCH compared to the [¹⁸F]FCH group was observed. FIG. 17 shows typical (0.5 mm) transverse PET image slices demonstrating accumulation of [¹⁸F]FCH and [¹⁸F]D4-FCH in human melanoma SKMEL-28 xenografts. In this mouse model, the tumor signal-to-background contrast was qualitatively superior in the [¹⁸F]D4-FCH PET images compared to [¹⁸F]FCH images. Both radiotracers had similar tumor kinetic profiles detected by PET (FIG. 17). The kinetics were characterized by rapid tumor influx with peak radioactivity at 1 min (FIG. 17). Tumor levels then equilibrated until 5 min followed by a plateau. The delivery and retention of [¹⁸F]D4-FCH were quantitatively higher than those for FCH (FIG. 17). The % ID/vox₆₀ for [¹⁸F]D4-FCH and [¹⁸F]FCH were 7.43±0.47 and 5.50±0.49, respectively (P=0.04). Because tumors often present with heterogeneous population of cells, another imaging variable that is probably less sensitive to experimental noise was exploited an average of the maximum pixel % ID/vox₆₀ across 5 slices (%IDvox₆₀ₘₐₓ). This variable was also significantly higher for [¹⁸F]D4-FCH(P=0.05; FIG. 17). Furthermore, tumor area under the time versus radioactivity curve (AUC) was higher for D4-FCH mice than FCH(P=0.02). Although the 30 min time point was selected for a more detailed analysis of tissue samples, the percentage of parent compound in plasma was consistently higher for [¹⁸F]D4-FCH compared to [¹⁸F]FCH at earlier time points. Regarding imaging, tumor uptake for both radiotracers was similar at the early (15 min) and late (60 min) time points (Supplementary Tablet). The earlier time points may be appropriate for pelvic imaging.

### Example 19

### Imaging Response to Treatment

Having demonstrated that [¹⁸F]D4-FCH was a more stable fluorinated-choline analog for in vivo studies, the use of this radiotracer to measure response to therapy was investigated. These studies were performed in a reproducible tumor model system in which treatment outcomes had been previously characterized, i.e. the human colon carcinoma xenograft HCT116 treated with PD0325901 daily for 10 days (Leyton, J., et al., “Noninvasive imaging of cell proliferation following mitogenic extracellular kinase inhibition by PD0325901”, Mol Cancer Ther 2008; 7(9):3112-21). Drug treatment led to tumor stasis (reduction in tumor size by only 12.2% at day 10 compared to the pretreatment group); tumors of vehicle-treated mice increased by 375%. Tumor [¹⁸F]D4-FCH levels in PD0325901-treated mice peaked at approximately the same time as those of vehicle-treated ones, however, there was a marked reduction in radiotracer retention in the treated tumors (FIG. 18). All imaging variables decreased after 10 days of drug treatment (P=0.05, FIG. 18). This indicates that [¹⁸F]D4-FCH can be used to detect treatment response even under conditions where large changes in tumor size reduction are not seen (Leyton, J., et al., “Noninvasive imaging of cell proliferation following mitogenic extracellular kinase inhibition by PD0325901”, Mol Cancer Ther 2008; 7(9):3112-21). To understand the biomarker changes, the intrinsic cellular effect of PD0325901 on D4-FCH-phosphocholine formation was examined by treating exponentially growing HCT116 cells in culture with PD0325901 for 24 h and measuring the 60-min uptake of [¹⁸F]D4-FCH in vitro. As shown in FIG. 18, PD0325901 significantly inhibited [¹⁸F]D4-FCH-phosphocholine formation in drug-treated cells demonstrating that the effect of the drug in tumors is likely due to cellular effects on choline metabolism rather than hemodynamic effects.

To understand further the mechanisms regulating [¹⁸F]D4-FCH uptake with drug treatment, changes in CHKA expression in PD0325901 and vehicle-treated tumors excised after PET scanning were assessed. A significant reduction in CHKA protein expression was seen in vivo at day 10 (P=0.03) following PD0325901 treatment (FIG. 19) indicating that reduced CHKA expression contributed to the lower D[¹⁸F]4-FCH uptake in drug-treated tumors. The drug-induced reduction of CHKA expression also occurred in vitro in exponentially growing cells treated with PD0325901.

### Example 20

### Statistics

Statistical analyses were done using the software GraphPad Prism version 4 (GraphPad). Between-group comparisons were made using the nonparametric Mann-Whitney test. Two-tailed P≦0.05 was considered significant.

All patents, journal articles, publications and other documents discussed and/or cited above are hereby incorporated by reference.

1. A compound of Formula (II): 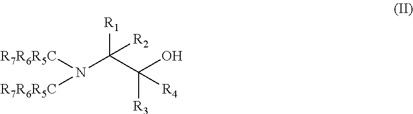
wherein:
R₁, R₂, R₃, and R₄ are each independently hydrogen or deuterium (D);
R₅, R₆, and R₇ are each independently hydrogen, R₈, —(CH₂)ₘR₈, —(CD₂)ₘR₈, —(CF₂)ₘR₈, —CH(R₈)₂, or —CD(R₈)₂;
R₈ is independently hydrogen, —OH, —CH₃, —CF₃, —CH₂OH, —CH₂F, —CH₂Cl, —CH₂Br, —CH₂I, —CD₃, —CD₂OH, —CD₂F, CD₂Cl, CD₂Br, CD₂I, or —C₆H₅; and
m is an integer from 1-4.

2. A compound according to claim 1 wherein:
R₁, R₂, R₃, and R₄ are each independently hydrogen;
R₅, R₆, and R₇ are each independently hydrogen, R₈, —(CH₂)ₘR₈, —(CD₂)ₘR₈, —(CF₂)ₘR₈, or —CD(R₈)₂;
R₈ is hydrogen, —OH, —CH₃, —CF₃, —CH₂OH, —CH₂F, —CH₂Cl, —CH₂Br, —CH₂I, —CD₃, —CD₂OH, —CD₂F, CD₂Cl, CD₂Br, CD₂I, or —C₆H₅; and
m is an integer from 1-4.

3. A compound according to claim 1 wherein:
R₁ and R₂ are each hydrogen;
R₃ and R₄ are each deuterium (D);
R₅, R₆, and R₇ are each independently hydrogen, R₈, —(CH₂)ₘR₈, —(CD₂)ₘR₈, —(CF₂)ₘR₈, or —CD(R⁸)₂;
R₈ is hydrogen, —OH, —CH₃, —CF₃, —CH₂OH, —CH₂F, —CH₂Cl, —CH₂Br, —CH₂I, —CD₃, —CD₂OH, —CD₂F, CD₂Cl, CD₂Br, CD₂I, or —C₆H₅; and
m is an integer from 1-4.

4. A compound according to claim 1 wherein:
R₁, R₂, R₃, and R₄ are each deuterium (D);
R₅, R₆, and R₇ are each independently hydrogen, R₈, —(CH₂)ₘR₈, —(CD₂)ₘR₈, —(CF₂)ₘR₈, or —CD(R₈)₂;
R₈ is hydrogen, —OH, —CH₃, —CF₃, —CH₂OH, —CH₂F, —CH₂Cl, —CH₂Br, —CH₂I, —CD₃,
—CD₂OH, —CD₂F, CD₂Cl, CD₂Br, CD₂I, or —C₆H₅; and
m is an integer from 1-4.

5. A compound according to claim 1 of Formula (IIa):

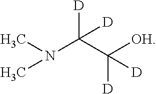

6. A compound of Formula (IIb) is provided: 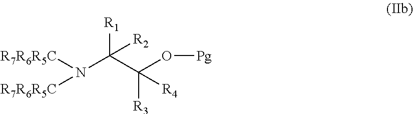
wherein:
R₁, R₂, R₃, and R₄ are each independently hydrogen or deuterium (D);
R₅, R₆, and R₇ are each independently hydrogen, R⁸, —(CH₂)ₘR₈, —(CD₂)ₘR₈, —(CF₂)ₘR₈, —CH(R⁸)₂, or —CD(R⁸)₂;
R₈ is independently hydrogen, —OH, —CH₃, —CF₃, —CH₂OH, —CH₂F, —CH₂Cl, —CH₂Br, —CH₂I, —CD₃, —CD₂OH, —CD₂F, CD₂Cl, CD₂Br, CD₂I, or —C₆H₅; and
m is an integer from 1-4; and
Pg is a hydroxyl protecting group.

7. A compound according to claim 6 wherein Pg is a p-methoxybenyzl (PMB), trimethylsilyl (TMS), or a dimethoxytrityl (DMTr) group.

8. A compound according to claim 6 wherein Pg is a p-methoxybenyzl (PMB) group.

9. A compound of Formula (IIc): 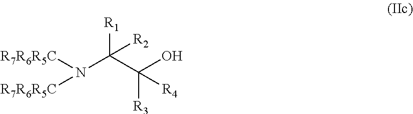
wherein:
R₁, R₂, R₃, and R₄ are each independently hydrogen or deuterium (D);
R₅, R₆, and R₇ are each independently hydrogen, R⁸, —(CH₂)ₘR₈, —(CD₂)ₘR₈, —(CF₂)ₘR₈, —CH(R⁸)₂, or —CD(R⁸)₂;
R₈ is independently hydrogen, —OH, —CH₃, —CF₃, —CH₂OH, —CH₂F, —CH₂Cl, —CH₂Br, —CH₂I, —CD₃, —CD₂OH, —CD₂F, CD₂Cl, CD₂Br, CD₂I, or —C₆H₅; and
m is an integer from 1-4;
with the proviso that when R₁, R₂, R₃, and R₄ are each hydrogen, R₅, R₆, and R₇ are each not hydrogen; and with the proviso that when R₁, R₂, R₃, and R₄ are each deuterium, R₅, R₆, and R₇ are each not hydrogen.

10. A compound according to claim 9 wherein:
R₁, R₂, R₃, and R₄ are each independently hydrogen;
with the proviso that R₅, R₆, and R₇ are each not hydrogen.

11. A compound according to claim 9 wherein:
R₁, R₂, R₃, and R₄ are each deuterium (D);
R₅, R₆, and R₇ are each not hydrogen.

12. A compound according to claim 9, wherein:
R₁ and R₂ are each hydrogen; and
R₃ and R₄ are each deuterium (D).
